(19) United States
(12) Patent Application Publication
(10) Pub. No.: US 2025/0312465 A1
(43) Pub. Date: Oct. 9, 2025

(54) STROMA PENETRATING THERAPEUTIC T-CELL ENGAGER FOR CANCER IMMUNOTHERAPY

(71) Applicant: Emory University, Atlanta, GA (US)

(72) Inventors: Lily Yang, Atlanta, GA (US); Lei Zhu, Atlanta, GA (US)

(21) Appl. No.: 19/170,816

(22) Filed: Apr. 4, 2025

Related U.S. Application Data
(60) Provisional application No. 63/574,371, filed on Apr. 4, 2024.

(51) Int. Cl. A61K 47/64; A61K 40/11; A61K 40/31; A61P 35/00; C12N 9/64; C12N 9/72

(52) U.S. Cl. CPC A61K 47/6425; A61K 40/11; A61K 40/31; A61P 35/00; C12N 9/6462; C12N 9/6491; C12Y 304/21073; C12Y 304/2408; C07K 2319/00

(57) ABSTRACT

The present disclosure relates to methods and compositions comprising a universal cell delivery ligand configured to enhance the intratumoral delivery and distribution of therapeutic immune cells by facilitating their penetration through dense stroma surrounding tumor cells.

### CROSS REFERENCE TO RELATED APPLICATIONS

This application claims the benefit of priority to U.S. Provisional Patent Application No. 63/574,371, filed on Apr. 4, 2024, the disclosure of which is expressly incorporated by reference herein in its entirety.

### STATEMENT REGARDING FEDERALLY SPONSORED RESEARCH

This invention was made with government support under grants R41CA247165-01A1, 3R41CA247165-01A1S1 and subcontract 0000053953 awarded by the National Institutes of Health. The government has certain rights in the invention.

### REFERENCE TO SEQUENCE LISTING

The sequence listing submitted on Jun. 20, 2025, as an.XML entitled “10029-216US1_ST26.xml” created on Apr. 7, 2025, and having a file size of 4,210 bytes is hereby incorporated by reference pursuant to 37 C.F.R. § 1.52(e)(5).

### FIELD

Disclosed herein are methods and compositions comprising a universal cell delivery ligand configured to enhance the intratumoral delivery of therapeutic immune cells by facilitating their penetration through dense stroma surrounding tumor cells.

### BACKGROUND

Cancer immunotherapy using adoptive T cell transfer has shown its promise in the treatment of hematological malignancies. Since 2017, The Food and Drug Administration (FDA) has approved six chimeric antigen receptor (CAR) T-cell therapies, including CD19 targeted BREYANZI® by Bristol Myers Squibb, KYMRIAH® (Novartis), TECARTUS® and YESCARTA® (Gilead's Kite), and BCMA targeted ABECMA® (Bristol Myers Squibb) and CARVYKTI® (Janssen Biotech). Those approved CAR T cells are used to treat acute lymphoblastic leukemia, B-cell lymphoma, and multiple myeloma. The development of CAR-T clinical trials has accelerated and over 1000 CAR-T clinical trials are registered on ClinicalTrials.gov in 2023. Up to date, 249 clinical trials (Phase 1/II) of CAR T in solid tumors have been registered in the National Institutes of Health (NIH) database, with more than 50 tumor targets have been investigated, such as glypican-3 (GPC3), disialoganglioside GD2 (GD2), epidermal growth factor receptor (EGFR); mucin 1 (MUC 1), human epidermal growth factor receptor 2 (HER2), prostate specific membrane antigen (PSMA), carcinoembryonic antigen (CEA), claudin 18.2 (CLDN18.2), mesothelin or mucin 16 (MUC16) on tumor cells. The majority of clinical trials in solid tumors are liver cancer, glioblastomas, pancreatic cancer, lung cancer, colorectal cancer, neuroblastoma and then prostate cancer, malignant pleural mesothelium, and head and neck cancer. Recently, the FDA approved lifileucel (AMTAGVI®) that uses patients' own tumor-infiltrating lymphocytes (TILs) to treat melanoma, which is the first cellular therapy to be approved for a solid tumor. Despite tremendous efforts, clinical trial results using antigen specific CAR-T cells in solid tumors have not yet yielded a good therapeutic response. The major obstacles include: 1) lack of suitable surface targets and highly heterogeneous tumor cells in solid tumors; 2) a low efficiency in CAR-T cell delivery into tumors; 3) tumor stromal barriers that limit migration of cytotoxic T cells to tumor cells; and 4) an immunosuppressive tumor stroma that inhibits proliferation and function of CAR-T cells. It has been shown that <1% of total delivered CAR T cells are able to enter into solid tumors and the majority of those CAR T cells are confined in the stroma. Since the cytotoxic function of T cells requires direct interaction of the TCR or CAR on T cells with tumor cells, the physical and immunosuppressive biological barriers in tumor stroma are the major challenges in adoptive T therapy in solid tumors. Additionally, there are serious side effects associated with CAR- T cell therapy, including cytokine release syndrome and neurological toxicity. Therefore, there is an urgent need to develop novel approaches to improve delivery efficiency and targetability of cytotoxic T cells in tumors and to overcome stroma barriers for effective cancer therapy. New methods and compositions for treating, inhibiting, reducing, and/or decreasing solid tumors are also needed.

### SUMMARY

Disclosed herein are methods and compositions of treating, inhibiting, reducing, decreasing, ameliorating, and/or preventing cancer related tumors in a subject in need thereof.

In some examples, disclosed herein is a universal cell delivery ligand, comprising a fusion protein conjugated with an inhibitor of C-X-C chemokine receptor type 4 (CXCR4), wherein the fusion protein comprises a first part and a second part, wherein the first part comprises an amino terminal fragment (ATF) of a receptor binding domain of urokinase plasminogen activator, and wherein the second part comprises a catalytic domain of matrix metalloproteinase-14 (mmp14).

In some examples, the inhibitor of CXCR4 comprises motixafortide (BL-8040), EMU050-derivatives or mavorixafor (X4P-001).

In some examples, the fusion protein comprises a sequence as set forth in SEQ ID NO: 1 or a sequence having at least 95%, 98%, 99% or 99.8% identity thereto.

In some examples, the amino terminal fragment of the receptor binding domain of urokinase plasminogen activator comprises a sequence as set forth in SEQ ID NO: 2 or a sequence having at least 95%, 98%, 99% or 99.8% identity thereto; or the catalytic domain of mmp14 comprises a sequence as set forth in SEQ ID NO: 3 or a sequence having at least 95%, 98%, 99% or 99.8% identity thereto.

In some examples, the inhibitor of CXCR4 binds to a CXCR4 receptor on at least one type of immune cell. In some examples, at least one type of immune cell comprises a T cell, a natural killer (NK) cell, or any combination thereof; wherein the T cell comprises CAR T cell, tumor infiltrating lymphocyte (TIL) or γδ T cell.

In some examples, disclosed herein is a pharmaceutical composition, comprising a universal cell delivery ligand; and a plurality of immune cells, wherein the universal cell delivery ligand comprises a fusion protein conjugated with an inhibitor of CXCR4, wherein the fusion protein comprises a first part and a second part, wherein the first part comprises an amino terminal fragment (ATF) of a receptor binding domain of urokinase plasminogen activator, and wherein the second part comprises a catalytic domain of mmp14.

In some examples, the plurality of immune cells comprise T cells, natural killer (NK) cells, or any combination thereof; wherein the T cells comprises CAR T cells, tumor infiltrating lymphocytes (TIL) or γδ T cells.

In some examples, the CAR T cells target glypican-3 (GPC3), disialoganglioside GD2 (GD2), epidermal growth factor receptor (EGFR); mucin 1 (MUC 1), human epidermal growth factor receptor 2 (HER2), prostate specific membrane antigen (PSMA), carcinoembryonic antigen (CEA), claudin 18.2 (CLDN18.2), mesothelin or mucin 16 (MUC16) on tumor cells.

In some examples, disclosed herein is a method of inhibiting an interaction between CXCR4 and C-X-C motif chemokine 12 (CXCL12) in a tumor, comprising administering to a subject a therapeutically effective amount of a universal cell delivery ligand conjugated to a plurality of immune cells, wherein the plurality of immune cells express CXCR4 on their surface, wherein the universal cell delivery ligand comprises a fusion protein conjugated with an inhibitor of CXCR4, wherein the fusion protein comprises a first part and a second part, wherein the first part comprises an amino terminal fragment (ATF) of a receptor binding domain of urokinase plasminogen activator, and wherein the second part comprises a catalytic domain of mmp14, thereby obtaining ATFmmp14/iCXCR4 ligand conjugated to plurality of immune cells expressing CXCR4 on their surface.

In some examples, the therapeutically effective amount of a universal cell delivery ligand with a plurality of immune cells is administered to the subject intravenously, intratumorally, intramuscularly, intradermally, or subcutaneously.

In some examples, the method increases mobility of the plurality of immune cells with the universal cell delivery ligand in tumor stroma as compared to a tumor stroma on which the method has not been performed.

In some examples, the subject is a human.

In some examples, the universal cell delivery ligand conjugated to the plurality of immune cells is administered at least one dose/day to the subject.

In some examples, the method further comprises administering at least one additional pharmaceutical agent to the subject. In some examples, the at least one additional pharmaceutical agent comprises pembrolizumab, 5-Fluorouracil (5-FU), leucovorin (folinic acid), irinotecan, or oxaliplatin.

In some examples, the tumor is a solid tumor. In some examples, the solid tumor comprises pancreatic cancer or colon cancer.

In some examples, disclosed herein is a method for making the universal cell delivery ligand, comprising conjugating a CXCR4 inhibitor to a linker molecule to form a linked inhibitor; and conjugating the linked inhibitor to a fusion protein comprising the amino terminal fragment (ATF) of a receptor binding domain of urokinase plasminogen activator and the catalytic domain of matrix metalloproteinase14 (mmp14).

In some examples, the linker molecule comprises succinimidyl 4-(N20 maleimidomethyl) cyclohexane-1-carboxylate (SMCC) or NHS-PEG8-Maleimide. In some examples, the SMCC forms one or more disulfide bonds to the CXCR4 inhibitor and the fusion protein.

In some examples, disclosed herein is a method of treating a subject with cancer, comprising isolating a plurality of immune cells from the peripheral blood of the subject with cancer, wherein the plurality of immune cells are T cells; engineer the T cells with a chimeric antigen receptor (CAR), thereby obtaining CAR T cells, wherein the CAR T cells express CXCR4 cell surface receptor; incubating a universal cell delivery ligand to CAR T cells, thereby obtaining universal cell delivery ligand bound CAR T cells, wherein the universal cell delivery ligand comprises a fusion protein conjugated with an inhibitor of CXCR4; wherein the fusion protein comprises a first part and a second part; wherein the first part comprises an amino terminal fragment (ATF) of a receptor binding domain of urokinase plasminogen activator; and wherein the second part comprises a catalytic domain of mmp14, wherein the CXCR4 inhibitor of the universal cell delivery ligand binds to the CAR T cell expressing CXCR4 cell surface receptor; and administering a therapeutically effective dose of the universal cell delivery ligand bound CAR T cells to the subject with cancer.

In some examples, the universal cell delivery ligand is incubated for about 30-60 minutes with the CAR T cells before administration.

In some examples, the therapeutically effective dose of the universal cell delivery ligand comprises 1 mg/dose, 2 mg/dose, 5 mg/dose or 10 mg/dose.

In some examples, the method further comprises administering at least one additional pharmaceutical agent to the subject. In some examples, the at least one additional pharmaceutical agent comprises pembrolizumab, 5-Fluorouracil (5-FU), leucovorin (folinic acid), irinotecan, or oxaliplatin.

In some examples, disclosed herein is a pharmaceutical composition for increased intratumoral delivery of cancer therapy, comprising a universal cell delivery ligand; and a pharmaceutically acceptable carrier, wherein the universal cell delivery ligand comprises a fusion protein conjugated with an inhibitor of CXCR4; wherein the fusion protein comprises a first part and a second part; wherein the first part comprises an amino terminal fragment (ATF) of a receptor binding domain of urokinase plasminogen activator; and wherein the second part comprises a catalytic domain of mmp14.

In some examples, the pharmaceutically acceptable carrier comprises a nanoparticle or a liposome.

Universal cell delivery ligand (ATFmmp14/iCXCR4) bound immune cells have exceptionally increased targeted delivery and therapy efficacy in tumor models as seen in FIGS. 3B, 6A and 7B.

uPAR targeted MMP14 active targeting ligand conjugated with inhibitors of CXCR4 (iCXCR4 or iCR4), for example, BL-8040 (peptidomimetic) or X4P-001 (small molecule) is a novel drug and cell delivery system with a unique design and biological properties. At present, there is no other similar cell delivery ligand that has multiple biological functions of targeting tumor endothelial cells for improving intratumoral delivery, binding stroma fibroblasts to facilitate migration, and digesting fibrillar collagens and other extracellular matrix for promoting stroma penetration, and targeting tumor cells to enhance the interactions of the CAR on T cells with tumor cell targets. Conjugation of iCXCR4 to the ATFmmp14 ligand adds an inhibitory effect on CXCR4-CXCL12 signal mediated immunosuppression on CAR T cells, which not only enhances cytotoxicity of CAR T cells but also markedly increases persistence of CAR T cells in tumors. ATFmmp14/iCXCR4 is an excellent T cell delivery ligand that has the potential to overcome clinical challenges of low delivery efficiency, stroma trapping and immunosuppression.

### BRIEF DESCRIPTION OF THE DRAWINGS

The accompanying figures, which are incorporated in and constitute a part of this specification, illustrate several examples described below.

FIG. 1 shows uPAR targeted and MMP14 active fusion ligand (ATFmmp14) conjugated with CXCR4 inhibitor, a peptide mimetic BL-8040 or a small molecule, X4P-001 as a universal, targeted, stroma-penetrating T cell delivery ligand for cancer immunotherapy in solid tumors.

FIGS. 2A, 2B, 2C, 2D, 2E, 2F and 2G show development and characterization of ATFmmp14/BL ligand (AMP/BL) and AMP/BL-bound δδ and MUC16 CAR-T cells. FIG. 2A shows protocol of direct chemical conjugation of ATFmmp14 to T cells. BL-8040 is a 14-mer peptide with modified amino acid sequences: BL-4F-Benzoyl-Arg-Arg-{2-Naph-Ala}-Cys-Tyr-{Cit}-Lys-Lys-Pro-Tyr-Arg-{Cit}-Cys-Arg NH2 (Disulfide bridge: Cys4-Cys13). NH2 group of two Lys at the C-terminal are good conjugation sites for ATFmmp14. A SMCC linker was used to conjugate BL-8040 to ATFmmp14. FIG. 2B shows flow cytometry detected 91% of MUC16 CAR T cells expressed CXCR4. FIG. 2C shows protocol of the production of the AMP/BL T cell delivery ligand and its binding to T cells. FIG. 2D shows binding and in vitro stability of NIR 830 labeled AMP/BL (purple NIR fluorescence) on CAR T cells. Live/dead assay determined the high viability of the ligand-bound CAR T cells at 136 hrs. FIG. 2E shows BL-8040(iCXCR4) mediated binding of AMP/BL to CXCR4 on the CAR T cells was competed off using a high concentration of non-labeled BL-8040. FIG. 2F shows CM Dil dye labeled (red), Fluor-405-ATFmmp14 (blue)/BL bound CAR T cells isolated 24 hrs after intratumoral injection in a PANC II PDX tumor. Human CD3+ (green) and CM Dil CAR T cells (red) retained the binding of AMP/BL (blue). FIG. 2G shows conjugation or binding of ATFmmp14 to γδ T or CAR T cells did not affect cell viability at 9 to 196 hrs using Live and dead assay (viable cells: green, dead: red).

FIGS. 3A and 3B show cytotoxicity of ATFmmp14 conjugated or AMP/BL-bound γδ or MUC16 CAR-T cells on human pancreatic cancer cell lines. FIG. 3A shows ligand-conjugated γδ T cells showed a strong cytotoxic effect on pancreatic cancer cells. FIG. 3B shows ATFmmp14 conjugated, or AMP/BL bound MUC16 CAR T cells had a strong cytotoxic effect on pancreatic cancer cells. There was a large difference in the levels of cytotoxicity among MUC16 CAR T cells derived from different health donors (donor numbers are shown). ATFmmp14 conjugated MUC16 CAR T cells showed stronger inhibitory effect compared to ATFmmp14 conjugated γδ T cells.

FIGS. 4A and 4B show targeted delivery of ATFmmp14 or AMP/BL-γδ or MUC16 CAR-T cells in pancreatic or colon PDX models. FIG. 4A shows optical imaging, whole body or ex vivo, detected targeted delivery into PDX tumors (arrows) and biodistribution in normal tissues (ex vivo imaging). Stronger signals detected in PDX tumors in the mice received ATFmmp14 or AMP/BL-γδ T cells than γδ T cell only. FIG. 4B shows improved intratumoral delivery of ATFmmp14-CAR T or AMP/BL-CAR T cells in pancreatic and colon PDX tumors.

FIGS. 5A, 5B, 5C, 5D, 5E, 5F and 5G show flow cytometry and histological analyses determined intratumorally delivered ATFmmp14 conjugated or bound MUC16 CAR T cells. FIGS. 5A and 5B show flow cytometry analysis of isolated cells from PDX tumors received i.v. delivery of 1×10⁷ of CAR T-, ATFmmp14-, AMP/BL-CAR T cells for 48 or 168 hrs. CAR T cells were determined as CD3+/MUC16cd+ cells. Percentages of the total delivered cells at 48 hrs are shown in red. Pink numbers are CAR T cell numbers in a total of 106 cells at 48 and 168 hrs. FIG. 5C shows the numbers of CAR T cells in tumors were further determined by immunofluorescence labeling using an anti-human CD3 antibody. Bar figure shows the mean CD3+ CAR T cells per 20× imaging field. FIGS. 5D and 5E show flow cytometry of isolated cells from two types of human pancreatic cancer patient derived xenograft (PDX) tumors (PANCII and PANCXXIV PDX tumors) 24 hrs following i.v. delivery of Celltrack CM Dil (red) labeled CAR T cells. Total delivered cells include CM Dil/CD3+ cells (upper) and CM Dil+ cells (lower). FIG. 5F shows the summary results of delivered CAR T cells in pancreatic cancer PDX models in the tumor, blood and spleen. FIG. 5G shows the detection of Celltrack dye labeled CAR or AMP/BL-CAR T cells (red) in tissue sections of tumors and normal organs 24 hrs after delivery.

FIGS. 6A and 6B show the effect of ATFmmp14- or AMP/iCR4-MUC16 CAR T cells on tumor growth and mouse survival in pancreatic cancer PDX models. FIG. 6A shows PANCII PDX model SCID mice bearing large s.c. PDX tumors (>300 mm³) received two i.v. injections of 5×10⁶ of MUC16 CAR T cells without or with ATFmmp14 or ATFmmp14/iCXCR4 at 15 and 80 days following PDX tumor implantation. Only ATFmmp14/iCXCR4 bound CAR T cell treated mouse group showed significant tumor growth inhibition. Mice treated with ATFmmp14-CAR T or AMP/iCR4-CAR T cells had significant improvement in the overall survival time. N=6 mice/group. FIG. 6B shows PANCXXIV PDX bearing mice were treated once with 5×10⁶ of ATFmmp14-or AMP/BL-MUC16 CAR T cells. N=4 mice/group.

FIGS. 7A and 7B show the histological characterization of pancreatic PDX tumors after MUC16 CAR T treatment. FIG. 7A shows H&E stained PDX tissue sections from FIG. 6A. AMP/BL-CAR T cell treated tumors had large tumor necrotic areas but did not affect normal tissues. FIG. 7B shows the immunofluorescence labeling. AMP/BL-CAR T cells significantly inhibited cell proliferation (Ki67+), reduced CK19+ tumor cells even at ˜90 days after treatment. All CAR T cell treated tumors had a marked decrease in FAP+ fibroblasts.

FIG. 8 shows the detection of MUC16 CAR T cells in the PDX tumors treated with AMP/BL-CAR T cells for 25 days. PDX tumors obtained in the efficacy study in FIG. 6A were analyzed for the level of MUC16 CAR T cells using immunofluorescence and an anti-human CD3 antibody. Yellow arrows: CAR T cells in tumor center bind to cancer cells in necrotic areas. Blue arrows: tumor areas with a low level of CAR T cells. Images: 10×, 20× lens

FIGS. 9A and 9B show the levels of MUC16 expression in human cancer tissues and pancreatic PDX tumors. FIG. 9A shows MUC16 gene expression in 7 common types of human solid tumors obtained from the TCGA data. FIG. 9B shows MUC16^{ecto} protein level in pancreatic cancer PDX tissues.

FIGS. 10A, 10B and 10C show the comparison of CAR T cell binding and cytotoxic effects of two types of iCXCR4. FIG. 10A shows a newly synthesized ATFmmp14/iCXCR T cell delivery ligand, AMP/X4P-001 (AMP/X4P), by conjugating a small molecule CXCR4inhibitor, X4P-001 mediated by an NHS-PEG8-Maleimide linker. FIG. 10B shows NIR 830 dye labeled AMP/BL or AMP/X4P bound to CAR T cells (Celltrack red) at a similar efficiency and stability. FIG. 10C shows a comparison of cytotoxicity of two ligand-bound MUC16 CAR T in PANCII PDX tumor cells using a cell proliferation assay. Percentage of survival cells is relative to the No-treatment control (100%).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular examples only and is not intended to be limiting.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “about” and “approximately” are defined as being “'close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genitourinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed.

A “composition” is intended to include a combination of active agent and another compound or composition, inert (for example, a detectable agent or label) or active, such as an adjuvant.

As used herein, the terms “determining,” “measuring,” and “assessing,” and “assaying” are used interchangeably and include both quantitative and qualitative determinations.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single-or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01.

“Substitution” refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “subject” or “host” refers to any individual who is the target of administration or treatment. The subject can be a vertebrate, for example, a mammal. Thus, the subject can be a human or veterinary patient. The term “patient” refers to a subject under the treatment of a clinician, e.g., physician. The subject can be either male or female.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder (e.g., a cancer), or a symptom of a disease or disorder. The terms “treating” and “treatment” can also refer to reduction in severity and/or frequency of symptoms, elimination of symptoms and/or underlying cause, and improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g. cancer). Therapeutically effective and prophylactically effective amounts of a given therapeutic agent will typically vary with respect to factors such as the type and severity of the disorder or disease being treated and the age, gender, and weight of the subject. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

The term “endogenous” refers to any material that is derived from or produced within an organism, cell, tissue, or system.

The term “exogenous” refers to any material introduced from or produced outside of an organism, cell, tissue, or system.

The term “expression” refers to the transcription and/or translation of a particular nucleotide sequence driven by a promoter.

The term “transfer vector” refers to a composition of matter that comprises an isolated nucleic acid and can be used to deliver the isolated nucleic acid to the interior of a cell. Many vectors are known in the art, including but not limited to linear polynucleotides, polynucleotides associated with ionic or amphiphilic compounds, plasmids, and viruses. Thus, the term “transfer vector” includes an autonomously replicating plasmid or virus. The term should also be construed to also include non-plasmid and non-viral compounds that facilitate transfer of nucleic acids into cells, such as poly lysine compounds, liposomes, and the like. Examples of viral transfer vectors include, but are not limited to, adenoviral vectors, adeno-associated viral vectors, retroviral vectors, lentiviral vectors, and the like.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Expression vector”, “expression construct”, “plasmid”, or “recombinant DNA construct” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity, when necessary, through the use of an inducer, in some systems however the protein may be expressed constitutively. As described herein, Bacillus subtilis can be used as the host for protein production, but other cell types may also be used.

In some examples, numbers expressing quantities of ingredients, properties such as molecular weight, reaction conditions, and so forth, used to describe and claim certain examples of the present disclosure are to be understood as being modified in some instances by the term “about.” In some examples, the term “about” is used to indicate that a value includes the standard deviation of the mean for the device or method being employed to determine the value. In some examples, the numerical parameters set forth in the written description and attached claims are approximations that can vary depending upon the desired properties sought to be obtained by a particular embodiment. In some examples, the numerical parameters should be construed in light of the number of reported significant digits and by applying ordinary rounding techniques. Notwithstanding that the numerical ranges and parameters setting forth the broad scope of some examples of the present disclosure are approximations, the numerical values set forth in the specific examples are reported as precisely as practicable. The numerical values presented in some examples of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

The “tumor stroma” as used herein, is a critical component of the tumor microenvironment. It has crucial roles in supporting tumor initiation, progression, and metastasis by producing various growth factors, chemokines, and cytokines. Moreover, the dense collagen matrix of the tumor stroma is rich in cancer associated fibroblasts, which can contract and tighten the collagen network by secreting extracellular matrix associated with molecules and integrin dependent binding. Such a barrier not only physically prevents the access of therapeutic agents to reach tumor cells but also causes high interstitial fluid pressure to prevent the efficient discharge drugs and infiltration of immune cells in the tumor. Fibrotic tumor stroma is common in many stroma-rich human tumors, such as pancreatic, liver, colon, and triple negative breast cancers. There is experimental and clinical evidence that stroma-normalizing and therapeutic agents may synergize if administered in a specific sequence or combination. A tumor stroma with cells refers to the non-neoplastic supportive tissue associated with a tumor, comprising an extracellular matrix and one or more types of stromal cells. The “stromal cells” may include, but are not limited to, fibroblasts, immune cells (e.g., lymphocytes, macrophages), endothelial cells, and pericytes.

As used herein, “urokinase plasminogen activator receptor” (uPAR) is an important target for the development of targeted therapeutic agents. uPAR interacts with its ligand uPA to regulate matrix degradation, cell migration, metastasis, and angiogenesis. Thus, a high level of uPAR is found in many types of human cancers, such as breast, lung, pancreatic, ovarian, colon, and brain cancer. Specifically, uPAR is expressed in invasive tumor cells, active stromal fibroblasts and macrophages, and angiogenic tumor endothelial cells. To overcome stromal cells and extracellular matrix barriers, we developed a new generation of uPAR targeted ligand with the ability to break the fibrotic stromal barrier for improving drug delivery. This stroma penetrating ligand is a recombinant protein by fusing amino-terminal fragment (ATF) of uPA fused with the catalytic domain of matrix metalloproteinase-14 (ATFmmp14). As used herein, ATFmmp14 is also written as AMP.

As used herein, “MMP-14” is a membrane-type MMP that has a broad substrate specificity on collagen (I, II, &III), gelatin, vitronectin, cartilage, proteoglycans, and fibrillin. The catalytic domain of MMP-14 only breaks large collagen fibrils allowing nanoparticle/drugs to penetrate through but is unable to further activate MMP-2 and MMP-9 activity to digest collagens into small fragments. Therefore, conjugation of this recombinant ligand enables CAR T cells extravasation, interacts with stroma cells to accelerate migration, and degrades extracellular matrix to penetrate through dense stroma.

“CXCR4” is an alpha-chemokine receptor specific for CXCL12. CXCL12/CXCR4 signaling pathway plays critical roles in tumor initiation and progression by activating multiple signaling pathways, regulating cancer stem cells and promoting tumor metastases.

Recently preclinical and clinical investigations have shown that treatment with a CXCR4 antagonist inhibited tumor growth and reduced the development of metastases. CXCL12/CXCR4 signal in tumor associated fibroblasts has been associated with cytotoxic T-lymphocyte exclusion. Inhibition of CXCR4 increased T cell infiltration and reduced fibroblasts and collagen content.

As used herein, “BL-8040” (Motixafortide) is a small synthetic peptide CRCX4 inhibitor. BL-8040 treatment increased CD8+ effector T-cell filtration in cancer tissues, decreased myeloid derived suppressor cells in the tumor microenvironment, and decreased circulating immunosuppressive Tregs cells. CXCR4 is expressed at a high level in different types of cytotoxic T cells.

In some examples, as used herein “inhibitors of CXCR4” (iCXCR4 or iCR4) like BL-8040 (peptidomimetic) or X4P-001 or EMU050-derivatives (small molecule derivatives) are conjugated to ATFmmp14, and serves as a ready-to-use linker to bind to T cells via CXCR4. As used herein, ATFmmp14 (AMP) conjugated with BL-8040 is denoted in some examples as AMP/BL.

uPAR targeted MMP14 fusion protein conjugated with iCXCR4 (BL-8040, X4P-001 or EMU050) is a novel drug and cell delivery system with a unique design and biological properties. As used herein, ATFmmp14 (AMP) conjugated with X4P-001 is denoted in some examples as AMP-X4P. At present, there is no other similar cell delivery ligand that has multiple biological functions of targeting tumor endothelial cells for improving intratumoral delivery, binding stroma fibroblasts to facilitate migration, and digesting fibrillar collagens and other extracellular matrix for promoting stroma penetration, and targeting tumor cells to enhance the interactions of the CAR on T cells with tumor targets. Conjugation of iCXCR4 to the ATFmmp14 ligand adds an inhibitory effect on CXCR4-CXCL12 signal mediated immunosuppression on CAR T cells, which not only enhances cytotoxicity of CAR T cells but also markedly increases persistence of CAR T cells in tumors. Thus, ATFmmp14/iCXCR4 is an excellent T cell delivery ligand that has the potential to overcome clinical challenges of low delivery efficiency, stroma trapping and immunosuppression.

Currently, there is no other similar delivery technologies under preclinical and clinical development. Although bispecific T-cell engagers (BiTEs), a group of bispecific antibodies that target 1 tumor antigen and 1 immune-related molecule, have attracted great attention for improving delivery and interaction of T cells with tumor cells, those bi-specific molecules only serves as linkers to bring T cells to tumor cells. Anti-CD3-antibody can activate T cell activity. To date, only a bispecific T-cell engager, blinatumomab (BCMA/CD3 BiTE) has received the FDA approval for a hematological cancer. Recently, the FDA has accepted and granted priority review for Amgen's biologics license application (BLA) for Tarlatamab to treat adult patients with advanced small cell lung cancer (SCLC) after disease progression on platinum-based chemotherapy. Tarlatamab is a delta-like ligand 3 (DLL3) targeting bispecific T-cell engager therapy that is under review and if approved, is the first approved bispecific antibody to treat a solid tumor. However, DLL3 is expressed on the cell surface of small cell lung cancer cells. There is no mechanism for tumor endothelial targeting to increase intratumoral delivery and tumor stroma penetration and modulation of immunosuppressive tumor microenvironment. Similarly, various bispecific T-cell engagers that are under preclinical or clinical developments, either as a T cell engager to motivate the endogenous T cells or delivery of CAR T cells, target tumor antigens that mainly express in tumor cells and could not bring T cells to pass through dense stroma.

In some examples, ATFmmp14/iCXCR4 conjugates demonstrated a superior effect on increasing in the efficiency of intratumoral delivery, distribution, and persistence of CAR T cells in pancreatic cancer patient derived xenograft (PDX) tumors. A significant enhancement in therapeutic efficacy of ATFmmp14/iCXCR4 has also been demonstrated in the PDX tumor models.

In some examples, the cancerous region or tumor comprises one or more of a benign tumor, a pre-metastatic tumor, or a malignant tumor.

In some examples, the stromal region comprises one or more of connective tissue, blood vessels, and inflammatory cells.

“Adoptive T cell therapy (ACT)” is a therapeutic modality that involves manipulating the cancer patient's own T cells to confer anti-tumor activity. This is accomplished by harvesting, ex vivo activation, modification and amplification, and re-infusion into the patient. The goal of the process is to generate efficient and cancer antigen-specific T cell immunity. Tumor-associated antigens can be divided into 3 major classes:
1. antigens are present in healthy tissue, but are overexpressed in tumors, usually because they confer a growth advantage on cancer cells;
2./1neoantigen produced by somatic mutation in cancer cells;
3. cancer germline antigens are proteins expressed on germline cells, located in immune-privileged sites, and thus are not susceptible to autoimmune T cell targeting.

The first successful application of ACT was the use of Tumor Infiltrating Lymphocytes (TIL), which produced a clinical response in approximately 50% of patients with malignant melanoma (Topallian et al., 1988). The wide applicability of this modality of treatment is hampered by the necessity of surgery to access the tissue from which the TIL is isolated, the difficulty of successfully isolating and amplifying the TIL, and the difficulty of reproducing similar results in other malignancies. Gene transfer-based strategies were developed to overcome T cell lineages specific to tumors is immune tolerance. These methods redirect T cells to effectively target tumor antigens through stable transduction transfer affinity optimized T Cell Receptors (TCRs) or synthetic Chimeric Antigen Receptors (CARs) based on retroviruses or lentiviruses. CAR T cells represent the most widely characterized ACT platform. CAR T cells are autologous T cells that have been reprogrammed to target surface-expressed cancer-associated antigens, typically by including single chain antibody variable fragments (scFv). These binding domains are fused to the costimulatory domain and CD3 zeta chain and subsequently transfected into autologous T cells using viral or non-viral transduction procedures. Upon binding to its cognate antigen, CAR T phosphorylates the immunoreceptor tyrosine-based activation motif (ITAM) within the CD3 zeta chain. This serves as an initiating T cell activation signal and is critical for CAR T-mediated tumor antigen lysis. At the same time, scFv binding also stimulates a fused common mimicry domain (usually CD28 or 4-1BB) that provides important amplification and survival signals. In 2017, the FDA approved two CD 19-directed CAR T cell approaches for treating patient patients with childhood Acute Lymphoblastic Leukemia (ALL) or diffuse large B-cell lymphoma (DLBCL), respectively: tisagenlecucel (Kymriah™) And sirolimus-acarbon (axicabagene cilel) (Yescata™) (CBER, 2017 a; CBER 2017 b). The former was also approved by the FDA in 2018 for the treatment of patients with relapsed/refractory DLBCL. Despite this activity in hematologic malignancies, CAR T cells fail to produce significant clinical efficacy against solid cancers, primarily due to T cell depletion and very limited persistence. By using only 1 of the 6 different T cell receptor subunits (CD3 zeta chain) in combination with a costimulatory domain, the CAR operates outside of the native TCR signaling complex. Failure to initiate and utilize a complete TCR response may be said to be a major potential factor preventing the success of CAR T cells in solid tumor indications.

As used herein, the term “linker molecule” refers to a bifunctional or multifunctional chemical moiety capable of covalently joining two or more molecular entities, such as a small-molecule inhibitor and a protein or peptide, through reactive functional groups. In certain embodiments, the linker molecule provides spatial separation, flexibility, or controlled reactivity between conjugated components, while preserving or enhancing their respective biological activities.

In some examples, the linker molecule is used to conjugate a CXCR4 inhibitor to a fusion protein comprising the amino terminal fragment (ATF) of the receptor-binding domain of urokinase plasminogen activator and the catalytic domain of matrix metalloproteinase 14 (MMP14). The linker serves as a chemical bridge between these components, forming a universal cell delivery ligand with enhanced targeting or functional properties.

In certain embodiments, the linker molecule comprises succinimidyl 4-(N-maleimidomethyl) cyclohexane-1-carboxylate (SMCC) or NHS-PEGs-Maleimide, both of which are heterobifunctional crosslinkers. These linkers contain a N-hydroxysuccinimide (NHS) ester group that reacts with primary amines (e.g., lysine residues on proteins), and a maleimide group that selectively reacts with thiol groups (e.g., cysteine residues or thiolated inhibitors), thereby facilitating site-specific and stable covalent attachment.

In some examples, the SMCC linker forms one or more disulfide-stabilized or thioether bonds between the CXCR4 inhibitor and the fusion protein. The resulting linked inhibitor-fusion protein complex can exhibit enhanced targeting to CXCR4-positive cells and retain enzymatic or receptor-binding activity, suitable for use in targeted delivery, imaging, or therapeutic applications.

### Compositions

As disclosed herein, a universal cell delivery ligand, comprising a fusion protein conjugated with an inhibitor of C-X-C chemokine receptor type 4 (CXCR4), wherein the fusion protein comprises a first part and a second part, wherein the first part comprises an amino terminal fragment (ATF) of a receptor binding domain of urokinase plasminogen activator, and wherein the second part comprises a catalytic domain of matrix metalloproteinase-14 (mmp14). In some examples, the inhibitor of CXCR4 comprises motixafortide (BL-8040), EMU050 derivatives or mavorixafor (X4P-001). Also disclosed herein, in some examples, the fusion protein ATFmmp14 contains an amino acid sequence SEQ ID NO: 1 or a fragment thereof. In some examples, the catalytic domain of mmp14 comprises sequence SEQ ID NO: 3 or a sequence having at least 80%, 85%, 87%, 89%, 90%, 91%, 92%, 93%, 94%, 95%, 96%, 97%, 98%, 99% or 100% identity thereto. A “fragment” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some examples a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference amino acid sequence.

As disclosed herein, in some examples, the urokinase plasminogen activator comprises a fragment which includes but is not limited to the amino terminal fragment (ATF) of uPA receptor binding domain. As disclosed herein, in some examples the ATF comprises a sequence from about amino acid number 1 to about amino acid number 68 of SEQ ID NO: 1 or a sequence having at least 95%, 98%, 99% or 99.8% identity thereto.

In some examples, disclosed herein is a pharmaceutical composition comprising a universal cell delivery ligand (ATFmmp14/iCXCR4), in combination with a pharmaceutically acceptable carrier.

Pharmaceutical compositions of the present disclosure may comprise a pharmaceutical composition for increased intratumoral delivery of cancer therapy, comprising a universal cell delivery ligand; and a pharmaceutically acceptable carrier, OR a universal cell delivery ligand; and a plurality of immune cells, wherein the universal cell delivery ligand comprises a fusion protein conjugated with an inhibitor of CXCR4; wherein the fusion protein comprises a first part and a second part; wherein the first part comprises an amino terminal fragment (ATF) of a receptor binding domain of urokinase plasminogen activator; and wherein the second part comprises a catalytic domain of mmp14.

As described herein, and one or more pharmaceutically or physiologically acceptable carriers, diluents, or excipients. Such compositions may comprise buffers, such as neutral buffered saline, phosphate buffered saline, and the like; carbohydrates such as glucose, mannose, sucrose or dextran, mannitol; a protein; polypeptides or amino acids, such as glycine; an antioxidant; chelating agents such as EDTA or glutathione; adjuvants (e.g., aluminum hydroxide); and a preservative. The pharmaceutical compositions of the present disclosure may be formulated for intravenous administration.

The pharmaceutical compositions of the present disclosure may be administered in a manner suitable for the disease to be treated (or prevented). The amount and frequency of administration will be determined by factors such as the condition of the patient and the type and severity of the patient's disease, although appropriate dosages may be determined by clinical trials.

In some examples, disclosed herein is a method for making the universal cell delivery ligand, comprising conjugating a CXCR4 inhibitor to a linker molecule to form a linked inhibitor; and conjugating the linked inhibitor to ATFmmp14.

In some examples, the linker molecule comprises succinimidyl 4-(N20 maleimidomethyl) cyclohexane-1-carboxylate (SMCC) or NHS-PEG8-Maleimide. In some examples, the SMCC forms one or more disulfide bonds to the CXCR4 inhibitor and the ATFmmp14.

Tagless ATFmmp14 may be produced using bacterial expression systems (e.g. Bacillus subtilis). ATFmmp14 produced in this manner has high binding affinity and MMP14 activity as well as large scale production of endotoxin-free ligands.

In some examples, disclosed herein is a universal cell delivery ligand, comprising a fusion protein conjugated with an inhibitor of C-X-C chemokine receptor type 4 (CXCR4), wherein the fusion protein comprises a first part and a second part, wherein the first part comprises an amino terminal fragment (ATF) of a receptor binding domain of urokinase plasminogen activator, and wherein the second part comprises a catalytic domain of matrix metalloproteinase-14 (mmp14). In some examples, the inhibitor of CXCR4 comprises motixafortide (BL-8040), EMU050-derivatives or mavorixafor (X4P-001).

In some examples, the fusion protein comprises a sequence as set forth in SEQ ID NO: 1 or a sequence having at least 95%, 98%, 99% or 99.8% identity thereto. In some examples, the catalytic domain of mmp14 comprises a sequence as set forth in SEQ ID NO: 3 or a sequence having at least 95%, 98%, 99% or 99.8% identity thereto.

In some examples, the inhibitor of CXCR4 binds to a CXCR4 receptor on at least one type of immune cell. In some examples, at least one type of immune cell comprises a T cell, a natural killer (NK) cell, or any combination thereof; wherein the T cell comprises CAR T cell, tumor infiltrating lymphocyte (TIL) or γδ T cell.

In some examples, disclosed herein is a pharmaceutical composition, comprising a universal cell delivery ligand; and a plurality of immune cells, wherein the universal cell delivery ligand comprises a fusion protein conjugated with an inhibitor of CXCR4, wherein the fusion protein comprises a first part and a second part, wherein the first part comprises an amino terminal fragment (ATF) of a receptor binding domain of urokinase plasminogen activator, and wherein the second part comprises a catalytic domain of mmp14.

ATFmmp14/iCXCR4 is added to the activated T cells 10, 20, 30, 40, 50 or 60 minutes before the administration of ATFmmp14/iCXCR4 conjugated activated T cells back into the subject. The concentration of ATFmmp14/iCXCR4 can be 1 mg/1×10⁸ activated immune cells, 2 mg/1×10⁸ activated immune cells, 3 mg/1×10⁸ activated immune cells, 4 mg/1×10⁸ activated immune cells, 3 mg/2×10⁸ activated immune cells, 4 mg/3×10⁸ activated immune cells, 1 mg/5×10⁸ activated immune cells, 2 mg/5×10⁸ activated immune cells, 5 mg/5×10⁸ activated immune cells, 1.5 mg/7.5×10⁸ cells, 2.5 mg/1.5×10⁸ cells, or 3.5 mg/2.5×10⁸ cells.

In some examples, the plurality of immune cells comprise T cells, natural killer (NK) cells, or any combination thereof; wherein the T cells comprises CAR T cells, tumor infiltrating lymphocytes (TIL) or γδ T cells.

In some examples, the CAR T cells target glypican-3 (GPC3), disialoganglioside GD2 (GD2), epidermal growth factor receptor (EGFR); mucin 1 (MUC 1), human epidermal growth factor receptor 2 (HER2), prostate specific membrane antigen (PSMA), carcinoembryonic antigen (CEA), claudin 18.2 (CLDN18.2), mesothelin or mucin 16 (MUC16) on tumor cells.

In some examples, disclosed herein is a method of inhibiting an interaction between CXCR4 and C-X-C motif chemokine 12 (CXCL12) in a tumor, comprising administering to a subject a therapeutically effective amount of a universal cell delivery ligand bound to a plurality of immune cells, wherein the plurality of immune cells express CXCR4 on their surface, wherein the universal cell delivery ligand comprises a fusion protein conjugated with an inhibitor of CXCR4.

In some examples, the pharmaceutical compositions/1 further comprises at least one additional pharmaceutical agent to the subject. In some examples, the at least one additional pharmaceutical agent comprises pembrolizumab, 5-Fluorouracil (5-FU), leucovorin (folinic acid), irinotecan, or oxaliplatin.

The pharmaceutical compositions provided herein can be used alone or in combination with conventional treatment regimen such as surgery, radiation, chemotherapy, and/or bone marrow transplantation (autologous, syngeneic, allogeneic or unrelated).

In some examples, at least one or more chemotherapeutic agents may be administered in addition to a pharmaceutical composition comprising an immunogenic therapy. In some examples, one or more chemotherapeutic agents may belong to different classes of chemotherapeutic agents.

### Methods of Treatment

In some examples, disclosed herein is a method of treating, inhibiting, reducing, decreasing, ameliorating, and/or preventing cancer in a subject, comprising administering to the subject a therapeutically effective dose of a pharmaceutical composition universal cell delivery ligand bound to an activated immune cell, wherein the universal cell delivery ligand conjugated to the activated immune cell comprises a fusion protein conjugated with an inhibitor of C-X-C chemokine receptor type 4 (CXCR4), wherein the fusion protein comprises a first part and a second part, wherein the first part comprises an amino terminal fragment (ATF) of a receptor binding domain of urokinase plasminogen activator, and wherein the second part comprises a catalytic domain of matrix metalloproteinase-14 (mmp14).

In some examples, the method provides at least a 50% percent decrease in tumor cells from baseline. In some examples, the method provides at least a 10%, 20%, 25%, 30%, 35%, 40%, 45%, 50%, 60%, 70%, 75%, 80%, 85%, 90%, 95%, or 98% decrease in tumor cells from baseline. In some embodiments, the method provides about a 10-20%, 10-25%, 15-30%, 15-35%, 20-40%, 20-45%, 25-50%, 30-60%, 35-70%, 50-60%, 50-75%, 60-90%, 70-90%, 80-90%, 80-95%, 80-98%, 85-98%, 90-98%, or 95-98% decrease in tumor cells from baseline.

In some examples, the subject has a solid tumor. Solid tumors generally comprise an abnormal mass of tissue that typically does not include cysts or liquid areas.

In some examples, the patient has a resectable solid tumor, meaning that the patient's tumor is deemed susceptible to being removed by surgery. In other embodiments, the patient has an unresectable solid tumor, meaning that the tumor has been deemed not susceptible to being removed by surgery, in whole or in part.

Also provided herein are methods of treating a subject having a tumor/cancer. The present disclosure provides methods of treatment comprising ATFmmp14/iCXCR4 conjugated or bound T cells. Methods of treating diseases (such as cancer) are provided. The method may comprise administering to the subject an effective amount of a pharmaceutical composition comprising ATFmmp14/iCXCR4 bound T cells.

In some examples, the method increases mobility of the plurality of immune cells with the universal cell delivery ligand in tumor stroma as compared to a tumor stroma on which the method has not been performed. In some examples, the subject is a human.

In some examples, the universal cell delivery ligand bound to the plurality of immune cells is administered at least one dose/day to the subject.

In some examples, the method further comprises administering at least one additional pharmaceutical agent to the subject. In some examples, the at least one additional pharmaceutical agent comprises pembrolizumab, 5-Fluorouracil (5-FU), leucovorin (folinic acid), irinotecan, or oxaliplatin.

In some examples, the universal cell delivery ligand is incubated for about 30-60 minutes with the CAR T cells before administration.

In some examples, the therapeutically effective dose of the universal cell delivery ligand comprises 1 mg/dose, 2 mg/dose, 5 mg/dose or 10 mg/dose.

In some examples, the universal cell delivery ligand has a concentration of about 1 mg/treatment to about 5 mg/treatment before administration.

In some examples, disclosed herein is a method of treating a subject with cancer, comprising isolating a plurality of immune cells from the peripheral blood of the subject with cancer, wherein the plurality of immune cells are T cells; engineer the T cells with a chimeric antigen receptor (CAR), thereby obtaining CAR T cells, wherein the CAR T cells express CXCR4 cell surface receptor; incubating a universal cell delivery ligand to CAR T cells, thereby obtaining universal cell delivery ligand bound CAR T cells, wherein the universal cell delivery ligand comprises a fusion protein conjugated with an inhibitor of CXCR4; wherein the fusion protein comprises a first part and a second part; wherein the first part comprises an amino terminal fragment (ATF) of a receptor binding domain of urokinase plasminogen activator; and wherein the second part comprises a catalytic domain of mmp14, wherein the CXCR4 inhibitor of the universal cell delivery ligand binds to the CAR T cell expressing CXCR4 cell surface receptor; and administering a therapeutically effective dose of the universal cell delivery ligand bound CAR T cells to the subject with cancer.

In some examples, a method of treating a subject having a tumor comprises administering to the subject a pharmaceutical composition disclosed herein. In some examples, the method is a method of making a stroma penetrating universal cell delivery ligand to address low delivery efficiency of cancer therapy, wherein the method comprises administering to a subject in need thereof a pharmaceutical composition disclosed herein. In some examples, the method is a method of inducing a cytotoxic response of immune cells targeted to tumor cells, wherein the method comprises administering to a subject in need thereof a pharmaceutical composition disclosed herein. In some examples, the method is a method to inhibit CXCL12/CXCR4 signaling in T cells induced by tumor cells or to inhibit T cell exhaustion by tumor cells.

In some examples, the subject has cancer, wherein the cancer is selected from the group consisting of: carcinomas, lymphomas, blastomas, sarcomas, leukemias, squamous cell carcinomas, lung carcinomas (including small-cell lung carcinoma, non-small cell lung carcinoma, lung adenocarcinoma, and lung squamous cell carcinoma), peritoneal carcinomas, hepatocellular carcinomas, gastric carcinomas (gastric cancer) or stomach carcinomas (stomach cancer), pancreatic carcinomas, glioblastomas, cervical carcinomas, ovarian carcinomas, liver carcinomas, bladder carcinomas, liver carcinomas, breast carcinomas, colon carcinomas, melanomas, endometrial or uterine carcinomas, salivary gland carcinomas, kidney carcinomas (kidney) or kidney (renal cancer), liver carcinomas, prostate carcinomas, vulval carcinomas, thyroid carcinomas, liver carcinomas, head and neck carcinomas, colorectal carcinomas, rectal carcinomas, soft tissue sarcomas, kaposi's sarcomas, B-cell lymphomas (including low-grade/follicular non-hodgkin's lymphoma (NHL), Small Lymphocytic (SL) NHL, medium-grade/follicular NHL, medium-grade diffuse NHL, B-cell lymphoma, Higher immunoblastic NHL, higher lymphoblastic NHL, higher small non-dividing cell NHL, giant tumor NHL, mantle cell lymphoma, AIDS-related lymphoma and waldenstrom's macroglobulinemia), Chronic Lymphocytic Leukemia (CLL), Acute Lymphoblastic Leukemia (ALL), myeloma, hairy cell leukemia, chronic myeloblastic leukemia and post-transplant lymphoproliferative disorder (PTLD), abnormal vascular proliferation associated with nevus destructor, edema, meglumine syndrome (Meigs' syndrome) and combinations thereof.

In some examples, the method further comprises administering at least one additional therapeutic agent or modality. In some examples, the at least one additional therapeutic agent or modality is surgery, checkpoint inhibitors, antibodies or fragments thereof, chemotherapeutic agents, radiation, vaccines, small molecules, T cells, vectors and APCs, polynucleotides, oncolytic viruses, or any combination thereof. In some examples, the at least one additional therapeutic agent is an anti-PD-1 agent and an anti-PD-L1 agent, an anti-CTLA-4 agent, or an anti-CD 40 agent. In some examples, the additional therapeutic agent is administered prior to, concurrently with, or subsequent to the administration of the pharmaceutical composition disclosed herein.

In some examples, the additional therapeutic agent is administered prior to, concurrently with, or subsequent to the administration of the pharmaceutical composition disclosed herein.

In some examples, the method of treating a subject with cancer prior to administration of the ATFmmp14/iCXCR4 bound T cells. Cancer treatments may include chemotherapy, immunotherapy, targeted agents, radiation therapy, and high dose corticosteroids. The method can include administering chemotherapy to the subject, including lymphodepleting chemotherapy with a high dose of a myeloablative agent. In some examples, the method comprises administering to the subject a preconditioning agent, such as a lymphocyte depleting agent or a chemotherapeutic agent, such as cyclophosphamide, fludarabine, or a combination thereof, prior to the first or subsequent agent. For example, the pretreatment agent can be administered to the subject at least 2 days prior to the first or subsequent dose, such as at least 3, 4, 5, 6, 7, 8, 9, or 10 days prior. In some examples, the pretreatment agent is administered to the subject no more than 10 days prior to the first or subsequent dose, such as no more than 9, 8, 7, 6, 5, 4, 3, or 2 days prior.

The universal cell delivery ligand (ATFmmp14/iCXCR4) can be introduced into immune cells like activated T cells, e.g., γδ T cells, CAR T cells, or tumor infiltrating T lymphocytes (TIL) that have been expanded in ex vivo culture, e.g., incubating the solution carrying the universal cell delivery ligand and letting it bind with T cell cultures. The universal cell delivery ligand attaches the activated T cells via the iCXCR4 domain, which binds to CXCR4 present on activated T cells. A subject (e.g., a human) receives an initial administration of ATFmmp14/iCXCR4 bound T cells of the disclosure, and one or more subsequent administrations of ATFmmp14/iCXCR4 bound T cells of the disclosure, wherein the one or more subsequent administrations are administered at about 2 to 4 weeks after the previous administration. A subject (e.g., a human) may be administered more than one administration of ATFmmp14/iCXCR4 bound T cells of during the treatment cycle, e.g. the second dose at 2 to 4 weeks after the first dose. A subject (e.g., a human subject) may receive more than one cycle of ATFmmp14/iCXCR4 bound T cells, and the time between each cycle is about 2 to 4 weeks after the previous administration.

When an “immunologically effective amount”, “anti-tumor effective amount”, “tumor inhibiting effective amount”, or “therapeutic amount” is indicated, the physician can determine the precise amount of the composition of the present disclosure to be administered, taking into account the individual differences in age, weight, tumor size, extent of infection or metastasis, and condition of the patient (subject). In general, it can be said that a pharmaceutical composition comprising ATFmmp14/iCXCR4 as a universal T cell delivery ligand that can be a commercial pharmaceutical product to be used in the clinic to deliver T cells and other immune cells with CXCR4 expression. The cells can be administered by using infusion techniques well known in immunotherapy (see, e.g., Rosenberg et al, New Eng.J.of Med.319:1676,1988).

It may be desirable to administer ATFmmp14/iCXCR4 bound, activated immune cells to a subject, followed by re-drawing blood (or performing an apheresis), wherein activated immune cells can be T cells, activating T cells from the blood and intratumoral infiltrating T cells according to the present disclosure, and re-infusing the patient with these activated and expanded T cells. This process may be performed multiple times every few weeks. T cells can be activated from 10 cc to 400 cc of blood draw. T cells can be activated from 20 cc, 30 cc, 40 cc, 50 cc, 60 cc, 70 cc, 80 cc, 90 cc, or 100 cc of blood taken.

In some examples, the method of treatment comprises one or more rounds of leukocyte depletion prior to CAR T cell transplantation and performing surgical operation. Leukopheresis may include the collection of Peripheral Blood Mononuclear Cells (PBMCs). Leukopheresis may include mobilization of PBMCs prior to collection. Alternatively, non-mobilized PBMCs may be collected. A large number of PBMCs can be collected from a subject in one round. Alternatively, the subject may undergo two or more rounds of leukapheresis. The volume of apheresis may depend on the number of cells required for transplantation. For example, 12-15 liters of non-mobilized PBMCs may be collected from a subject in a round. The number of PBMCs to be collected from a subject may be between 1×10⁸ to 5×10¹⁰ between individual cells. The number of PBMCs to be collected from a subject may be 1×10⁸, 5∴10⁸, 1×10⁹, 5×10⁹, 1×10¹⁰ or 5×10¹⁰ cells. The minimum number of PBMCs to be collected from a subject may be 1×10⁶ per kg subject weight. The minimum number of PBMCs to be collected from a subject may be 1×10⁶/kg, 5×10⁶/kg, 1×10⁷/kg, 5×10⁷/kg, 1×10⁸/kg, 5×10⁸ per kg subject weight.

In some examples, the subject may undergo leukapheresis, wherein leukocytes are collected, enriched, or removed ex vivo to select and/or isolate target cells, e.g., T cells. These T cell isolates can be expanded by methods known in the art and treated so that one or more universal cell delivery ligands of the disclosure can be introduced, thereby producing a pharmaceutical composition comprising a plurality of immune cells bound to universal cell delivery ligand of the disclosure. A subject in need thereof may then receive an infusion of the pharmaceutical composition of the present disclosure. The expanded cells can be administered before or after surgery.

Administration of the compositions of the invention may be carried out in any convenient manner, including by aerosol inhalation, injection, ingestion, infusion, implantation or transplantation. The pharmaceutical compositions described herein can be administered to a patient via the arterial, subcutaneous, intradermal, intratumoral, intranodal, intramedullary, intramuscular, by intravenous (i.v.) injection, or intraperitoneal route. The pharmaceutical compositions of the present disclosure may be administered to a patient by intravenous or intratumoral injections. The pharmaceutical compositions can be injected directly into a tumor, lymph node, or site of infection. In some examples, described herein are pharmaceutical compositions for parenteral administration comprising a solution of cells dissolved or suspended in an acceptable carrier, e.g., an aqueous carrier. A variety of aqueous carriers can be used, such as water, buffered water, 0.9% saline, 0.3% glycine, hyaluronic acid, and the like. These pharmaceutical compositions may be sterilized by conventional, well known sterilization techniques, or they may be filter sterilized. The compositions may contain pharmaceutically acceptable auxiliary substances as required to approximate physiological conditions such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like.

The dosage of such treatment to be administered to a subject will vary with the exact nature of the condition being treated and the recipient of the treatment. Dose scaling for human administration (scaling of usages) may be performed according to art-recognized practice. For example, for adult patients, the dose of alemtuzumab is typically in the range of 1 mg to about 100 mg, typically administered daily for a period of 1 to 30 days. A daily dose of 1 to 10 mg/day is preferred, although larger doses of up to 40 mg/day may be used in some cases (described in U.S. Pat. No. 6,120,766 hereby incorporated by reference in its entirety).

It is understood and herein contemplated that the disclosed treatment regimens can used alone or in combination with any anti-cancer therapy known in the art including, but not limited to Abemaciclib, Abiraterone Acetate, ABITREXATE® (Methotrexate), ABRAXANE® (Paclitaxel Albumin-stabilized Nanoparticle Formulation), ABVD, ABVE, ABVE-PC, AC, AC-T, ADCETRIS® (Brentuximab Vedotin), ADE, Ado-Trastuzumab Emtansine, ADRIAMYCIN® (Doxorubicin Hydrochloride), Afatinib Dimaleate, AFINITOR® (Everolimus), AKYNZEO® (Netupitant and Palonosetron Hydrochloride), ALDARA® (Imiquimod), Aldesleukin, ALECENSA® (Alectinib), Alectinib, Alemtuzumab, ALIMTA® (Pemetrexed Disodium), ALIQOPA® (Copanlisib Hydrochloride), ALKERAN™ for Injection (Melphalan Hydrochloride), ALKERAN™ Tablets (Melphalan), ALOXI® (Palonosetron Hydrochloride), ALUNBRIG® (Brigatinib), AMBOCHLORIN® (Chlorambucil), AMBOCLORIN® (Chlorambucil), Amifostine, Aminolevulinic Acid, Anastrozole, Aprepitant, AREDIA® (Pamidronate Disodium), ARIMIDEX® (Anastrozole), AROMASIN® (Exemestane),ARRANON® (Nelarabine), Arsenic Trioxide, ARZERRA® (Ofatumumab), Asparaginase Erwinia chrysanthemi, Atezolizumab, AVASTIN® (Bevacizumab), Avelumab, Axitinib, Azacitidine, BAVENCIO® (Avelumab), BEACOPP, BECENUM® (Carmustine), BELEODAQ® (Belinostat), Belinostat, Bendamustine Hydrochloride, BEP, BESPONSA® (Inotuzumab Ozogamicin), Bevacizumab, Bexarotene, BEXXAR® (Tositumomab and Iodine I 131 Tositumomab), Bicalutamide, BICNUR (Carmustine), Bleomycin, Blinatumomab, BLINCYTO® (Blinatumomab), Bortezomib, BOSULIF® (Bosutinib), Bosutinib, Brentuximab Vedotin, Brigatinib, BuMel, Busulfan, BUSULFEX® (Busulfan), Cabazitaxel, CABOMETYX® (Cabozantinib-S-Malate), Cabozantinib-S-Malate, CAF, CAMPATH® (Alemtuzumab), CAMPTOSAR® (Irinotecan Hydrochloride), Capecitabine, CAPOX, CARAC® (Fluorouracil-Topical), Carboplatin, CARBOPLATIN-TAXOL, Carfilzomib, CARMUBRIS® (Carmustine), Carmustine, Carmustine Implant, CASODEX®, (Bicalutamide), CEM, Ceritinib, CERUBIDINE® (Daunorubicin Hydrochloride), CERVARIX® (Recombinant HPV Bivalent Vaccine), Cetuximab, CEV, Chlorambucil, CHLORAMBUCIL-PREDNISONE, CHOP, Cisplatin, Cladribine, CLAFEN® (Cyclophosphamide), Clofarabine, CLOFAREX® (Clofarabine), CLOLAR® (Clofarabine), CMF, Cobimetinib, COMETRIQ® (Cabozantinib-S-Malate), Copanlisib Hydrochloride, COPDAC, COPP, COPP-ABV, COSMEGEN® (Dactinomycin), COTELLIC® (Cobimetinib), Crizotinib, CVP, Cyclophosphamide, CYFOS® (Ifosfamide), CYRAMZA® (Ramucirumab), Cytarabine, Cytarabine Liposome, CYTOSAR-UR (Cytarabine), CYTOXAN® (Cyclophosphamide), Dabrafenib, Dacarbazine, DACOGEN® (Decitabine), Dactinomycin, Daratumumab, DARZALEX® (Daratumumab), Dasatinib, Daunorubicin Hydrochloride, Daunorubicin Hydrochloride and Cytarabine Liposome, Decitabine, Defibrotide Sodium, DEFITELIO® (Defibrotide Sodium), Degarelix, Denileukin Diftitox, Denosumab, DEPOCYT® (Cytarabine Liposome), Dexamethasone, Dexrazoxane Hydrochloride, Dinutuximab, Docetaxel, DOXIL® (Doxorubicin Hydrochloride Liposome), Doxorubicin Hydrochloride, Doxorubicin Hydrochloride Liposome, DOX-SL® (Doxorubicin Hydrochloride Liposome), DTIC-DOME® (Dacarbazine), Durvalumab, EFUDEX® (Fluorouracil-Topical), ELITEK® (Rasburicase), ELLENCE® (Epirubicin Hydrochloride), Elotuzumab, ELOXATIN® (Oxaliplatin), Eltrombopag Olamine, EMEND® (Aprepitant), EMPLICITI® (Elotuzumab), Enasidenib Mesylate, Enzalutamide, Epirubicin Hydrochloride, EPOCH, ERBITUX® (Cetuximab), Eribulin Mesylate, ERIVEDGE® (Vismodegib), Erlotinib Hydrochloride, ERWINAZE® (Asparaginase Erwinia chrysanthemi), ETHYOL® (Amifostine), Etopophos ETOPOPHOS® (Etoposide Phosphate), Etoposide, Etoposide Phosphate, EVACET® (Doxorubicin Hydrochloride Liposome), Everolimus, EVISTA® (Raloxifene Hydrochloride), EVOMELA® (Melphalan Hydrochloride), Exemestane, 5-FU® (Fluorouracil Injection), 5-FU® (Fluorouracil-Topical), FARESTON® (Toremifene), FARYDAK® (Panobinostat), FASLODEX® (Fulvestrant), FEC, FEMARA® (Letrozole), Filgrastim, FLUDARA® (Fludarabine Phosphate), Fludarabine Phosphate, FLUOROPLEX® (Fluorouracil-Topical), Fluorouracil Injection, Fluorouracil-Topical, Flutamide, FOLEX® (Methotrexate), FOLEX PFS® (Methotrexate), FOLFIRI, FOLFIRI-BEVACIZUMAB, FOLFIRI-CETUXIMAB, FOLFIRINOX, FOLFOX, FOLOTYN® (Pralatrexate), FU-LV, Fulvestrant, GARDASIL® (Recombinant HPV Quadrivalent Vaccine), GARDASIL 9® (Recombinant HPV Nonavalent Vaccine), GAZYVAR (Obinutuzumab), Gefitinib, Gemcitabine Hydrochloride, GEMCITABINE-CISPLATIN, GEMCITABINE-OXALIPLATIN, Gemtuzumab Ozogamicin, GEMZAR® (Gemcitabine Hydrochloride), GILOTRIF® (Afatinib Dimaleate), GLEEVEC® (Imatinib Mesylate), GLIADEL® (Carmustine Implant), GLIADEL WAFER® (Carmustine Implant), Glucarpidase, Goserelin Acetate, HALAVEN® (Eribulin Mesylate), HEMANGEOL® (Propranolol Hydrochloride), HERCEPTIN® (Trastuzumab), HPV Bivalent Vaccine, Recombinant, HPV Nonavalent Vaccine, Recombinant, HPV Quadrivalent Vaccine, Recombinant, HYCAMTIN® (Topotecan Hydrochloride), HYDREA® (Hydroxyurea), Hydroxyurea, Hyper-CVAD, IBRANCE® (Palbociclib), Ibritumomab Tiuxetan, Ibrutinib, ICE, ICLUSIG® (Ponatinib Hydrochloride), IDAMYCIN® (Idarubicin Hydrochloride), Idarubicin Hydrochloride, Idelalisib, IDHIFA® (Enasidenib Mesylate), IFEX® (Ifosfamide), Ifosfamide, IFOSFAMIDUM® (Ifosfamide), IL-2 (Aldesleukin), Imatinib Mesylate, IMBRUVICA® (Ibrutinib), IMFINZI® (Durvalumab), Imiquimod, IMLYGIC® (Talimogene Laherparepvec), INLYTA® (Axitinib), Inotuzumab Ozogamicin, Interferon Alfa-2b, Recombinant, Interleukin-2 (Aldesleukin), INTRON A® (Recombinant Interferon Alfa-2b), Iodine I 131 Tositumomab and Tositumomab, Ipilimumab, IRESSA® (Gefitinib), Irinotecan Hydrochloride, Irinotecan Hydrochloride Liposome, ISTODAX® (Romidepsin), Ixabepilone, Ixazomib Citrate, IXEMPRA® (Ixabepilone), JAKAFI® (Ruxolitinib Phosphate), JEB, JEVTANA® (Cabazitaxel), KADCYLA® (Ado-Trastuzumab Emtansine), KEOXIFENER (Raloxifene Hydrochloride), KEPIVANCE® (Palifermin), KEYTRUDA®(Pembrolizumab), KISQALI® (Ribociclib), KYMRIAH® (Tisagenlecleucel), KYPROLIS® (Carfilzomib), Lanreotide Acetate, Lapatinib Ditosylate, LARTRUVO® (Olaratumab), Lenalidomide, Lenvatinib Mesylate, LENVIMA® (Lenvatinib Mesylate), Letrozole, Leucovorin Calcium, LEUKERAN® (Chlorambucil), Leuprolide Acetate, LEUSTATIN® (Cladribine), LEVULAN® (Aminolevulinic Acid), LINFOLIZIN® (Chlorambucil), LIPODOX® (Doxorubicin Hydrochloride Liposome), Lomustine, LONSURF® (Trifluridine and Tipiracil Hydrochloride), LUPRON® (Leuprolide Acetate), LUPRON DEPOT® (Leuprolide Acetate), LUPRON DEPOT-PED® (Leuprolide Acetate), LYNPARZA® (Olaparib), MARQIBO® (Vincristine Sulfate Liposome), MATULANE® (Procarbazine Hydrochloride), Mechlorethamine Hydrochloride, Megestrol Acetate, MEKINIST® (Trametinib), Melphalan, Melphalan Hydrochloride, Mercaptopurine, Mesna, MESNEX® (Mesna), METHAZOLASTONE® (Temozolomide), Methotrexate, METHOTREXATE LPF® (Methotrexate), Methylnaltrexone Bromide, MEXATE® (Methotrexate), MEXATE-AQ® (Methotrexate), Midostaurin, Mitomycin C, Mitoxantrone Hydrochloride, MITOZYTREX® (Mitomycin C), MOPP, MOZOBIL® (Plerixafor), MUSTARGEN® (Mechlorethamine Hydrochloride), MUTAMYCIN® (Mitomycin C), MYLERAN® (Busulfan), MYLOSAR® (Azacitidine), MYLOTARG® (Gemtuzumab Ozogamicin), NANOPARTICLE PACLITAXEL® (Paclitaxel Albumin-stabilized Nanoparticle Formulation), NAVELBINE® (Vinorelbine Tartrate), Necitumumab, Nelarabine, NEOSAR® (Cyclophosphamide), Neratinib Maleate, NERLYNX® (Neratinib Maleate), Netupitant and Palonosetron Hydrochloride, NEULASTA® (Pegfilgrastim), NEUPOGEN® (Filgrastim), NEXAVAR® (Sorafenib Tosylate), NILANDRON® (Nilutamide), Nilotinib, Nilutamide, NINLARO® (Ixazomib Citrate), Niraparib Tosylate Monohydrate, Nivolumab, NOLVADEX® (Tamoxifen Citrate), NPLATE® (Romiplostim), (Sonidegib), OEPA, Ofatumumab, OFF, Olaparib, Obinutuzumab, ODOMZO® Olaratumab, Omacetaxine Mepesuccinate, ONCASPAR® (Pegaspargase), Ondansetron Hydrochloride, ONIVYDE® (Irinotecan Hydrochloride Liposome), ONTAK® (Denileukin Diftitox), OPDIVO® (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, PARAPLAT® (Carboplatin), PARAPLATIN® (Carboplatin), Pazopanib Hydrochloride, PCV, PEB, Pegaspargase, Pegfilgrastim, Peginterferon Alfa-2b, PEG-INTRON® (Peginterferon Alfa-2b), Pembrolizumab, Pemetrexed Disodium, PERJETA® (Pertuzumab), Pertuzumab, PLATINOL® (Cisplatin), PLATINOL-AQ®(Cisplatin), Plerixafor, Pomalidomide, POMALYST® (Pomalidomide), Ponatinib Hydrochloride, PORTRAZZA® (Necitumumab), Pralatrexate, Prednisone, Procarbazine Hydrochloride, PROLEUKIN® (Aldesleukin), PROLIA® (Denosumab), PROMACTA® (Eltrombopag Olamine), Propranolol Hydrochloride, PROVENGE® (Sipuleucel-T), PURINETHOL® (Mercaptopurine), PURIXAN® (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, RELISTOR® (Methylnaltrexone Bromide), R-EPOCH, REVLIMID® (Lenalidomide), RHEUMATREX® (Methotrexate), Ribociclib, R-ICE, RITUXAN® (Rituximab), RITUXAN HYCELA® (Rituximab and Hyaluronidase Human), Rituximab, Rituximab and, Hyaluronidase Human,,Rolapitant Hydrochloride, Romidepsin, Romiplostim, RUBIDOMYCIN® (Daunorubicin Hydrochloride), RUBRACA® (Rucaparib Camsylate), Rucaparib Camsylate, Ruxolitinib Phosphate, RYDAPT® (Midostaurin), Sclerosol Intrapleural Aerosol (Talc), Siltuximab, Sipuleucel-T, SOMATULINE DEPOT® (Lanreotide Acetate), Sonidegib, Sorafenib Tosylate, SPRYCEL® (Dasatinib), STANFORD V, Sterile Talc Powder (Talc), STERITALC® (Talc), STIVARGA® (Regorafenib), Sunitinib Malate, SUTENT® (Sunitinib Malate), SYLATRON® (Peginterferon Alfa-2b), SYLVANT® (Siltuximab), Synribo SYNRIBO® (Omacetaxine Mepesuccinate), TABLOID® (Thioguanine), TAC, TAFINLAR® (Dabrafenib), TAGRISSO® (Osimertinib), Talc, Talimogene Laherparepvec, Tamoxifen Citrate, TARABINE PFS® (Cytarabine), TARCEVA® (Erlotinib Hydrochloride), TARGRETIN® (Bexarotene), TASIGNA® (Nilotinib), TAXOL® (Paclitaxel), TAXOTERE® (Docetaxel), TECENTRIQ® (Atezolizumab), TEMODAR® (Temozolomide), Temozolomide, Temsirolimus, Thalidomide, THALOMID® (Thalidomide), Thioguanine, Thiotepa, Tisagenlecleucel, TOLAK® (Fluorouracil--Topical), Topotecan Hydrochloride, Toremifene, TORISEL® (Temsirolimus), Tositumomab and Iodine I 131 Tositumomab, TOTECT® (Dexrazoxane Hydrochloride), TPF, Trabectedin, Trametinib, Trastuzumab, TREANDA® (Bendamustine Hydrochloride), Trifluridine and Tipiracil Hydrochloride, TRISENOX® (Arsenic Trioxide), TYKERB® (Lapatinib Ditosylate), UNITUXIN® (Dinutuximab), Uridine Triacetate, VAC, Vandetanib, VAMP, VARUBI® (Rolapitant Hydrochloride), VECTIBIX® (Panitumumab), VeIP, VELBAN® (Vinblastine Sulfate), VELCADE® (Bortezomib), VELSAR® (Vinblastine Sulfate), Vemurafenib, VENCLEXTAR (Venetoclax), Venetoclax, VERZENIO® (Abemaciclib), VIADUR® (Leuprolide Acetate), VIDAZAR (Azacitidine), Vinblastine Sulfate, VINCASAR PFS® (Vincristine Sulfate), Vincristine Sulfate, Vincristine Sulfate Liposome, Vinorelbine Tartrate, VIP, Vismodegib, VISTOGARD® (Uridine Triacetate), VORAXAZE®(Glucarpidase), Vorinostat, VOTRIENT® (Pazopanib Hydrochloride), VYXEOS®(Daunorubicin Hydrochloride and Cytarabine Liposome), WELLCOVORIN® (Leucovorin Calcium), XALKORI® (Crizotinib), XELODA® (Capecitabine), XELIRI, XELOX, XGEVA® (Denosumab), XOFIGO® (Radium 223 Dichloride), XTANDI® (Enzalutamide), YERVOY® (Ipilimumab), YONDELIS® (Trabectedin), ZALTRAP® (Ziv-Aflibercept), ZARXIO® (Filgrastim), ZEJULAR (Niraparib Tosylate Monohydrate), ZELBORAF® (Vemurafenib), ZEVALIN® (Ibritumomab Tiuxetan), ZINECARD® (Dexrazoxane Hydrochloride), Ziv-Aflibercept, ZOFRAN® (Ondansetron Hydrochloride), ZOLADEX® (Goserelin Acetate), Zoledronic Acid, ZOLINZA® (Vorinostat), ZOMETA® (Zoledronic Acid), ZYDELIG® (Idelalisib), ZYKADIA® (Ceritinib), and/or ZYTIGA® (Abiraterone Acetate). The treatment methods can include or further include checkpoint inhibitors including, but are not limited to antibodies that block PD-1 (such as, for example, Nivolumab (BMS-936558 or MDX1106), pembrolizumab, cemiplimab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B-and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### EXAMPLES

The following examples are set forth below to illustrate the compounds, systems, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art.

### Example 1: Cancer Immunotherapy Using Adoptive T Cell Transfer

Adoptive T cell therapy using isolated and engineered T cells that recognize and kill tumor cells has attracted great attention in cancer immunotherapy. Tumor infiltrating T lymphocytes (TIL) isolated from cancer patients have been expanded in vitro and then infused into the patients. Promising results have been reported in metastatic cancer patients, such as melanoma and lung cancer. CAR-T cell therapy that involves genetically engineered patient's autologous T cells or heathy donor's allogeneic T cells offers selective tumor cell killing ability. A CAR vector has an antigen-binding domain, signaling domains of the T cell receptor CD3ζ-chain, and additional costimulatory domains from T cell receptors, such as CD28, OX40, and 41BB. A number of CAR-T cells targeting tumor antigens, such as MUC1, MUC16, mesothelin, and CEA, have been developed and their anti-tumor effects have been evaluated in preclinical studies and clinical trials. Beside the above tumor antigen targeted CAR-T cell therapy, T cells expressing a heterodimeric γδ T cell receptor have shown promises in tumor cell killing. Cytotoxic effect of γδ T cells does not require co-stimulatory signals and is not limited to recognizing specific antigens. Cancer immunotherapy by systemic delivery of the above cytotoxic T cells has been under intensive preclinical and clinical developments.

The most success of CAR T cell therapy has largely been in hematological malignancies, which led to the FDA approval of anti-CD19 CAR T cells for the treatment of B-cell lymphoma and anti-BCMA CAR T cells for multiple myeloma. After three decades of clinical investigations, the FDA has recently approved lifileucel that uses patients' TILs to treat melanoma, which is the first cellular therapy to be approved for a solid tumor. Despite extensive studies, results of clinical trials using various CAR-T cells in solid tumors have not produced desired therapeutic response. It is well recognized that there are major challenges in CAR T cell therapy for solid tumor. Lack of efficiency in delivery of therapeutic T cells into solid tumors, perhaps, is the most significant hurdle. In solid tumors, there are multiple barriers that prevent CAR-T cells from being delivered into tumor tissues and reach tumor cells. Abnormal blood vessels limit T cell delivery into tumors. Dense tumor stromal cells and extracellular matrix create a physical barrier that prevents efficient infiltration of T cells through tumor stroma to reach tumor cells. Enriched tumor stroma has been found in many types of human cancers, such as pancreatic, colon, lung, liver, prostate, and triple negative breast cancer. For example, over 50% of a pancreatic tumor mass consists of tumor stroma. Pancreatic cancer has been classified as immune “cold” tumor due to lack of cytotoxic T cell infiltration. The presence of an immunosuppressive tumor microenvironment that often features immunosuppressive cells, including regulatory T cells, myeloid-derived suppressor cells (MDSCs), tumor promoting macrophages and fibroblasts, and inhibitory cytokines, such as IL-4, IL-6, IL-10, and TGFβ. Among a low number of intratumorally delivered CAR T cells, most are likely sequestered in a hostile immunosuppressive stroma that hinders CAR T cell function and persistence as well as promotes exhaustion of CAR T cells. Therefore, the development of novel approaches that improve intratumoral delivery and stroma-penetration of CAR T cells has great potential to enhance therapeutic response of solid tumors to CAR T therapy. Additionally, solid tumors exhibit considerable heterogeneity in their expression levels of tumor associated with antigen. CAR T cells are designed to target specific antigens on cancer cells, and the diversity of these antigens within a single tumor can lead to incomplete targeting and treatment resistance. There are also concerns of cytokine release syndrome and on-target, off-tumor toxicities of CAR T cells on normal tissues.

Various approaches have been developed to enhance specificity of CAR T cells, increase intratumoral delivery and trafficking, and overcome immunosuppression in the tumor stroma. Combining CAR T cells targeting multiple antigens or utilizing CAR T cells that express multiple antigens in CAR T constructs can address the challenge of tumor heterogeneity. Bi-specific T cell engagers with one binding to CD3 on T cells and the other targeting a tumor antigen to redirect endogenous T cells to tumor cells. MUC16 CAR T cells that secrete a T cell engager, such as a TCR mimic antibody that binds to tumor antigen WT1-epitope/HLA-A2, direct T cells to tumors, resulting in dual targeted CAR T cells with increased specificity and therapeutic efficacy.

Strategies to modify the immunosuppressive tumor microenvironment include co-administration of immune checkpoint inhibitors and cytokines/receptors to reprogram the microenvironment and make it more conducive to CAR T cell activity. Tumor-associated fibroblasts are the major type of stromal cells in solid tumors. Engineering CAR T cells to express chemokine receptors and cytokines that enhance migration into the tumor and enhance T cell function have been developed and evaluated in preclinical and clinical studies. A phase I clinical trial of the MUC16^{ecto}-specific CAR T cells modified to secrete IL-12 is ongoing in ovarian cancer patients (NCT02498912). Fibroblast activation protein (FAP)-CAR T cells can deplete FAP+ fibroblasts to enhance therapy response to mesothelin-targeted CAR T cells and anti-PD-1 antibody in a mouse pancreatic cancer model. Results from a clinical trial showed that a combination of CLDN6-specific CAR-T cells with CLDN6amplifying RNA vaccine enhanced therapeutic responses in cancer patients with relapsed solid tumors.

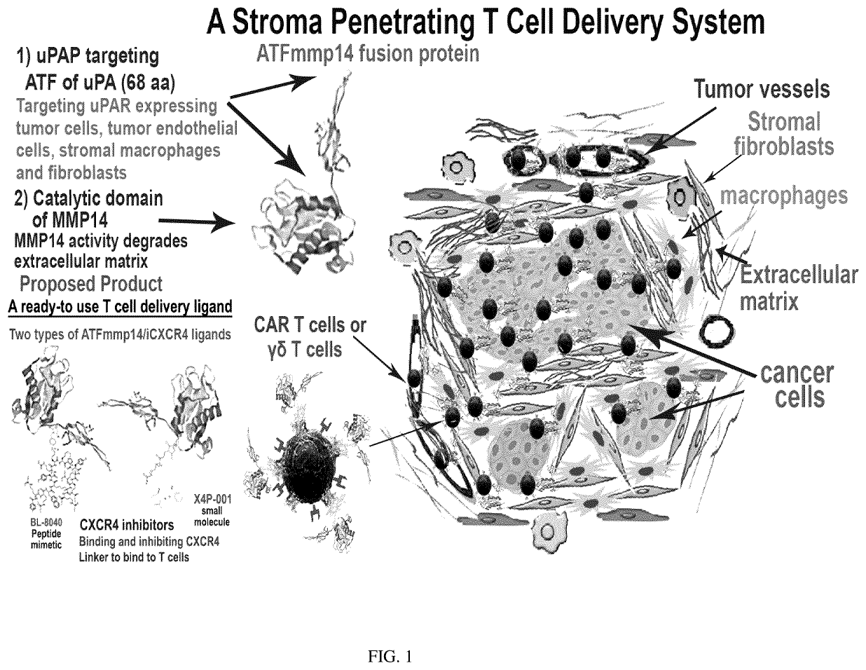

Addressing low delivery efficiency issue, a urokinase plasminogen activator receptor (uPAR)-targeted, stroma penetrating ligand is developed by fusing the amino terminal fragment (ATF) of uPA with the catalytic domain of MMP14 (ATFmmp14) (FIG. 1) (The composition of ATFmmp14 is described in PCT/US2014/069106, incorporated herein by reference). uPA binds to its receptor with a high affinity (KD of 0.1-1 nM). It is found that recombinant human ATFmmp14 retained a strong binding affinity to human uPAR with a KD of 17 nM. This fusion protein is expressed at a large amount in an endotoxin attenuated bacterial expressing system. Using this stroma penetrating ligand, several types of targeted nanoparticle/drugs are developed and have demonstrated significant enhancement in targeted delivery and the ability of migration through the tumor stroma to deliver nanoparticle/drug into tumor cells in pancreatic, breast and colon cancer patient derived PDX models. uPAR is highly expressed in tumor stromal and cancer cells in many types of cancer tissues but its expression is low in normal tissues. Angiogenic tumor endothelial cells express a high level of uPAR, but its expression level is very low in normal endothelial cells. Therefore, uPAR targeted delivery can lead to an increased accumulation in tumor tissues and facilitate extravasating T cells into tumors. Upon entering into tumor stroma, ATFmmp14 interacts with uPAR expressing tumor associated with fibroblasts and macrophages to promote migration of T cells in the tumor stroma. MMP14 (MT1-MMP) has a broad extracellular matrix substrate activity in large fibrillar collagens, fibronectin and laminin. Among all MMPs, MMP14 is only enzyme that has the degradative activity on basement membrane when the active catalytic domain is expressed in cells. MMP14 mediated degradation of surrounding matrix enables T cells to penetrate through stroma and across the basement membrane to reach ductal tumor cells (FIG. 1). uPAR is also highly expressed in invasive tumor cells. ATFmmp14 targeting to uPAR on tumor cells can enhance the binding of CAR T cells to tumor cell targets.

Recent advances in cancer immunotherapy by inhibiting CXCR4 and promoting recruitment of cytotoxic T cells in tumors. CXCR4 is an alpha-chemokine receptor specific to CXCL12. CXCL12/CXCR4 are highly expressed in tumor cells and stroma fibroblasts in many cancer types. CXCL12/CXCR4 signaling plays critical roles in tumor initiation and progression. Treatment with a CXCR4 antagonist inhibited tumor growth and reduced the development of metastases in mouse tumor models and clinical trials. Inhibition of CXCR4 also mobilized hematopoietic stem cells for transplantations in hematological malignancies. CXCL12/CXCR4 signal in tumor associated fibroblasts is associated with cytotoxic T cell exclusion. Inhibition of CXCR4 increased T cell infiltration and reduced fibroblasts and collagen content. BL-8040 (Motixafortide, BioLineRx Ltd) is a small synthetic peptide CXCR4 inhibitor that binds to CXCR4 with a high affinity (IC50=˜1 nM). The efficacy of BL-8040 in cancer therapy has been demonstrated in preclinical and clinical studies. Its effect on promoting mobilization of T cells from the bone marrow and lymph nodes into the periphery blood and tumors have also been demonstrated. Importantly, results of a clinical trial in metastatic pancreatic cancer patients showed that BL-8040 increased CD8+ T-cell filtration in cancers and decreased MDSC cells in the tumor microenvironment. The combination of BL-8040 with an anti-PD1 antibody enhanced therapeutic response and overall survival of cancer patients. At present, a clinical trial of combination Gemcitabine and Nab-Paclitaxel, Motixafortide, and Cemiplimab (PD1 antibody) in metastatic pancreatic cancer patients (NCT04543071) is ongoing. Small molecule CXCR4 inhibitors have also been developed, as X4P-001 (mavorixafor, such IC50=5.18 nM, X4 PHARMACEUTICALS®) and EMU050-derivatives (PCT/US2018/018973 is herein incorporated by reference for EMU116 derivatives, EMU050-derivatives). Clinical trial results showed that X4P-001 in combination with nivolumab enhanced antitumor immune responses in advanced renal cancer patients. Oral mavorixafor recently received FDA approval for the treatment of WHIM syndrome, an inherited immune deficiency. Since CXCR4 is expressed at a high level in cytotoxic T cells, BL-8040, X4P-001 or EMU050-derivatives has the potential to serve as a biologically active linker to connect ATFmmp14 to the surface of T cells via binding to CXCR4. BL-8040 was used as a linker in the development and characterization of ATFmmp14/iCXCR4 conjugates, which showed a superior effect on enhancing the dispersion, persistence, and delivery efficiency of CAR T cells in pancreatic PDX tumors. It has also been shown that ATFmmp14/iCXCR4 associated CAR T cells significantly improve treatment efficacy in PDX tumor models.

The novelties of the ATFmmp14/iCXCR4 T cell delivery ligand include: 1) the first universal T cell delivery system for improving intratumoral delivery of therapeutic T cells, including TIL, CAR T and γδ T cells; 2) the first T cell delivery system that enables T cell's ability of penetrating dense stroma to reach tumor cells; 3) blocking CXCR4/CXCL12 interaction to increase intratumoral delivery; 4) a novel design of ATFmmp14/iCXCR4 that binds to T cells and blocks the inhibitory effect of CXCL12 on T cell infiltration and activity; and 5) dual CAR T antigen and uPAR targeting facilitate the interactions of CAR T cells with their targets on tumor cells.

### Example 2: Methods and Materials

### Conjugation of the Selected iCXCR4 (X4P-001 or BL8040) to ATFmmp14R2 Ligand

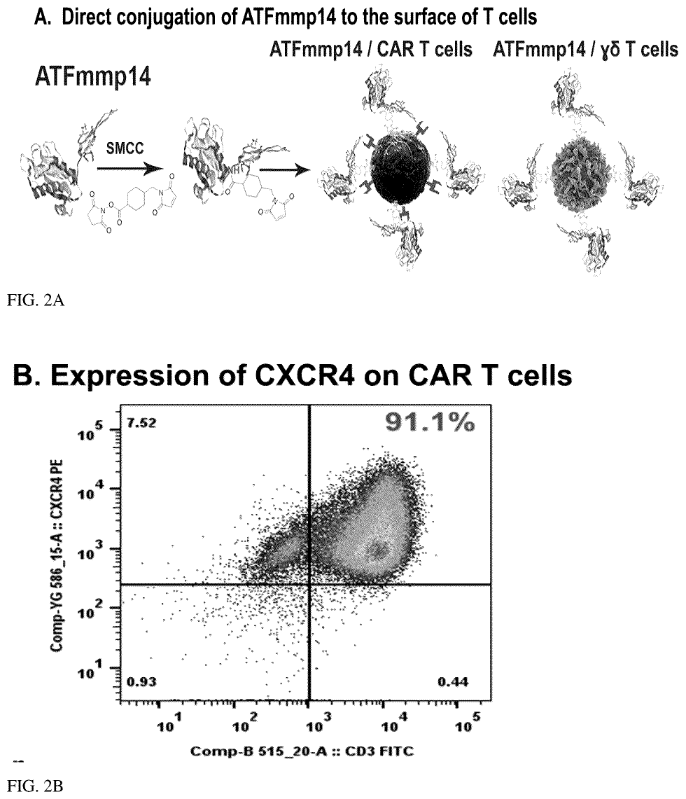

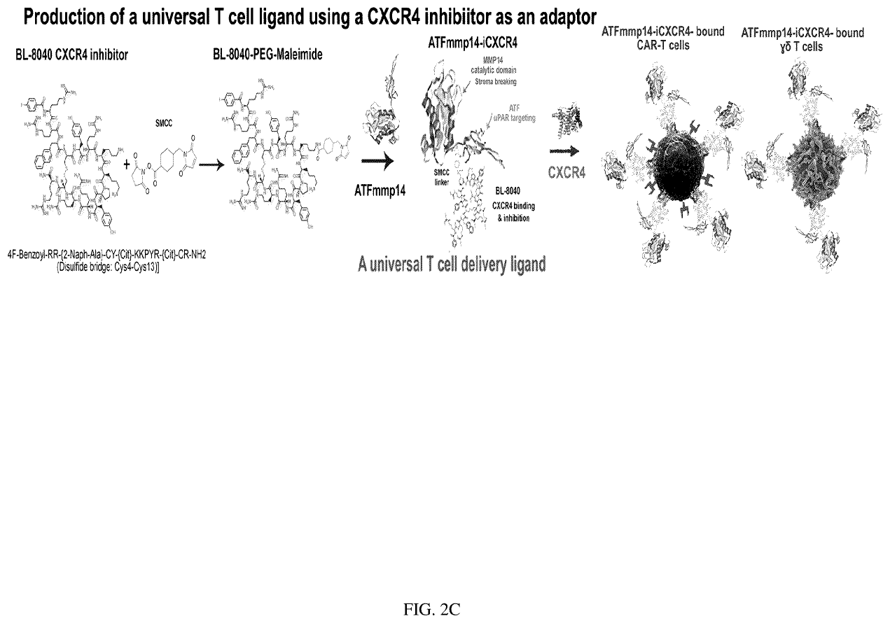

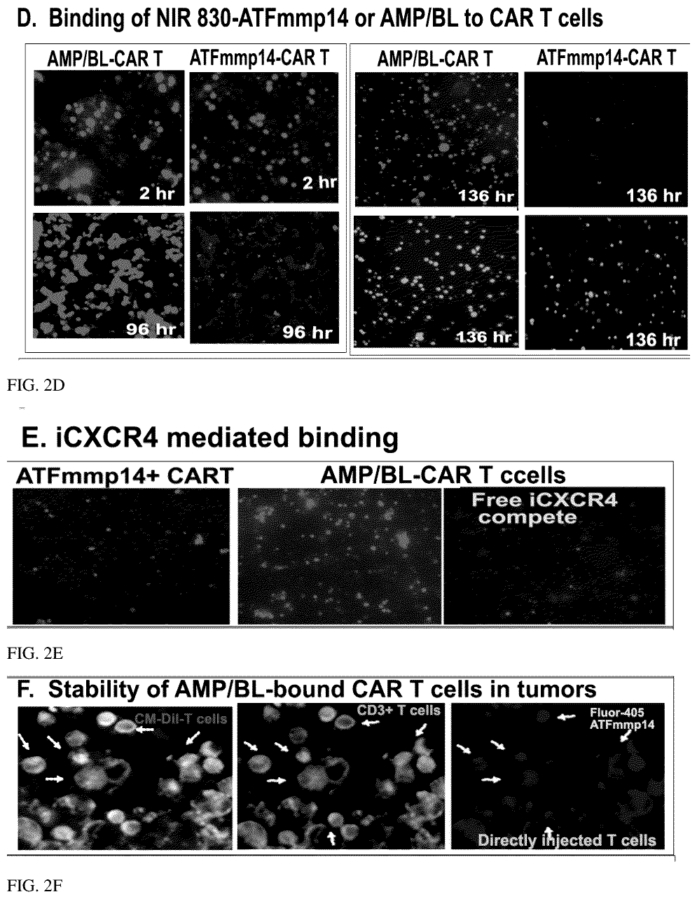

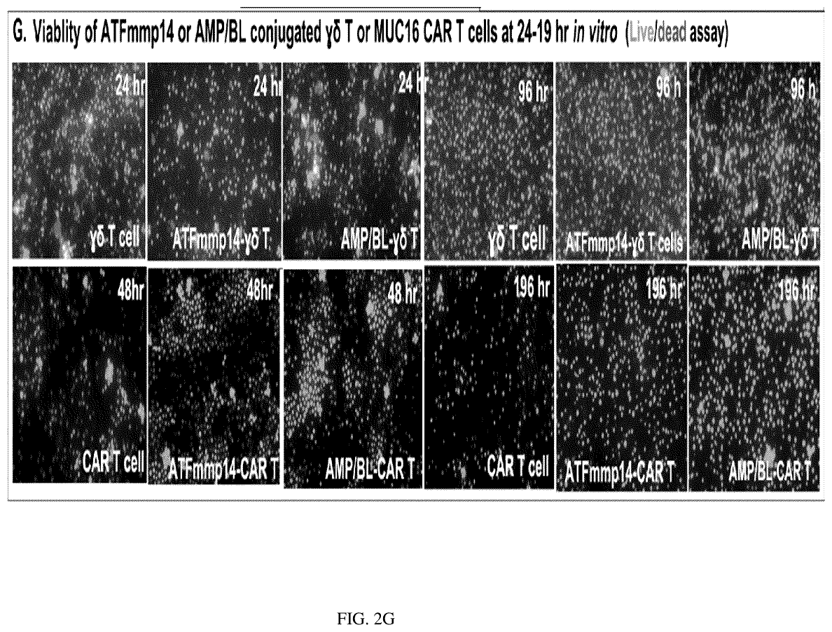

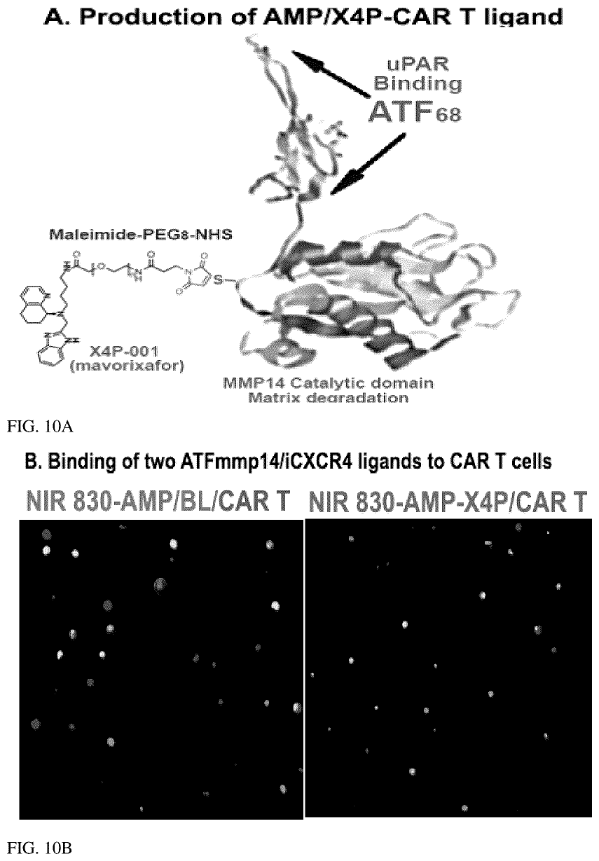

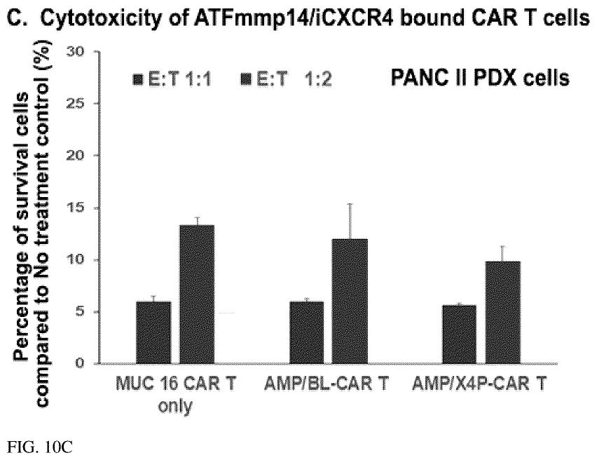

Considering the benefit of translation and commercialization, both BL-8040 and X4P-001 show similar targeted delivery and therapeutic efficacy. A method is developed for the conjugation of X4P-001 or BL-8040 to ATFmmp14R2 using 1:1 ratio (FIGS. 2 and 10). The binding of the ATF domain to uPAR and iCXCR4 to CXCR4 is confirmed by ELISA and competition assays in vitro. MMP14 activity of AMP/iCR4 is confirmed in vitro using a fluorescent MMP14 peptide substrate.

### Example 3: To Develop the Best Formulation of ATFmmp14 for Delivering Cytotoxic T Cells

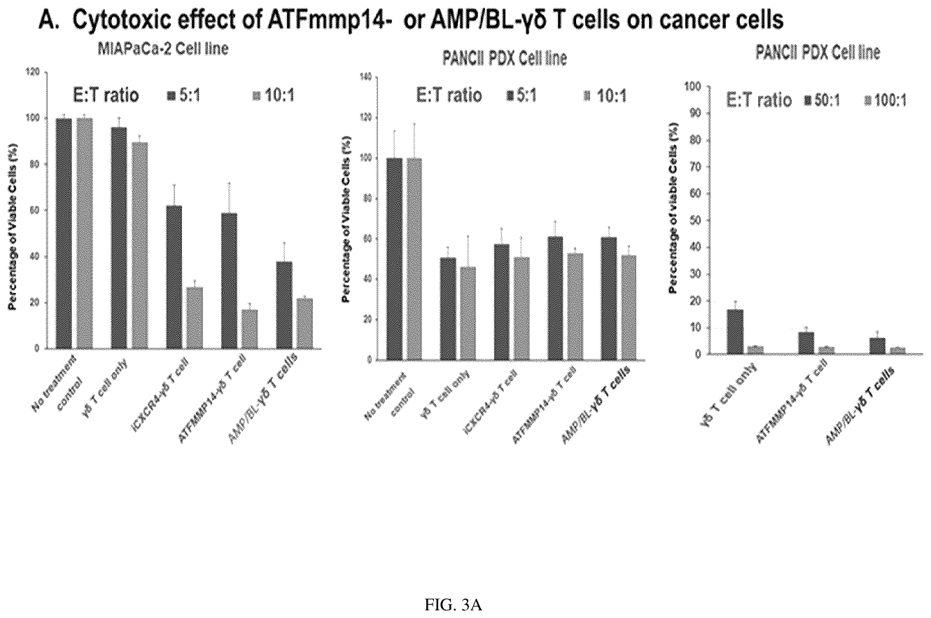

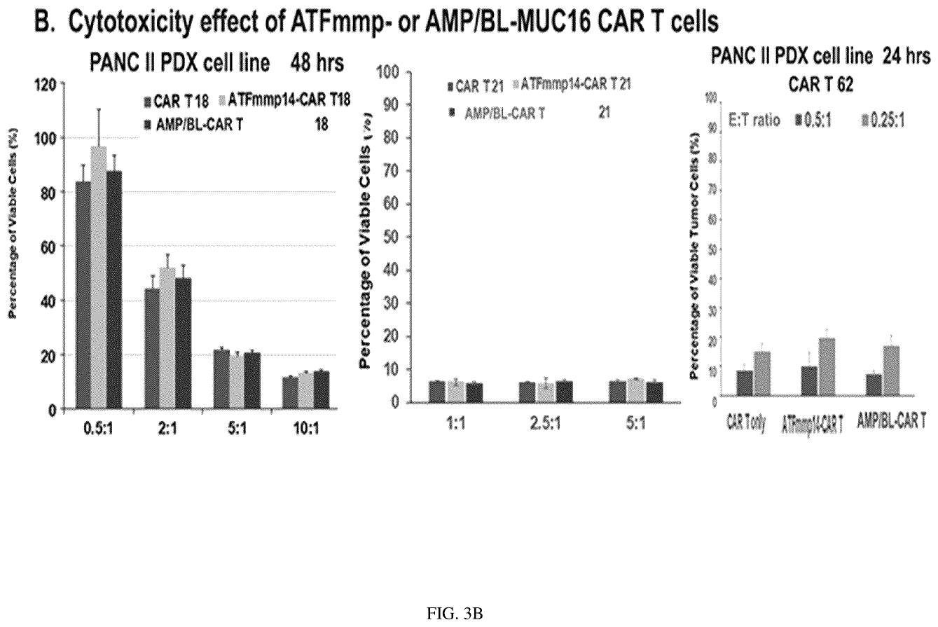

In order to determine the best formulation, two approaches were developed to produce uPAR targeted, stroma-breaking T cell delivery systems. The first method is a direct conjugation using SMCC linker mediated surface disulfide bond at a 50 μg ATFmmp14 to 5×10⁶ T cell ratio (FIG. 2A). Since about 91% CAR T cells express CXCR4 (FIG. 2B), the second method is to conjugate a SMCC linker to BL-8040 and then conjugate to ATFmmp14 via a disulfide bond, resulting in an ATFmmp14/iCXCR4 (AMP/BL) as a universal T cell delivery ligand that binds to CXCR4 highly expressed in activated T cells (FIG. 2C). In vitro analysis showed that although both NIR 830 labeled ATFmmp14 and AMP/BL were found on γδ and CAR T cells, a higher level of AMP/BL was seen on T cells than that of ATFmmp14 direct conjugation (FIG. 2D). Competition assay further supported the notion that the binding of AMP/BL to T cells was mediated by interaction with CXCR4 since a high concentration of un-labeled iCXCR4 could compete off the binding of NIR 830 dye labeled AMP/BL (FIG. 2E). In vivo stability of AMP/BL-CAR T cells was demonstrated after intratumoral injection of fluorescence labeled ligand and T cells since Fluor-405-ATFmmp14 was colocalized with CD3+ and CM Dil labeled CAR T cells after 24 hrs (FIG. 2F). Furthermore, conjugation of ATFmmp14 to γδ and CAR T cells did not affect cell viability (FIG. 2G). To determine whether the conjugation affects the interaction of T cells to tumor cells and cytotoxic function of γδ and MUC16 CAR-T cells. The cytotoxic effect on PANCII PDX-derived cell lines and MIAPaCa-2 pancreatic cancer was investigated. According to the findings, T cells' potent cytotoxicity against tumor cells was maintained when ATFmmp14 was conjugated to them or when AMP/BL was bound to them (FIG. 3).

### Example 4: To Determine the Effect of ATFmmp14 on Intratumoral Delivery and Activity of CAR-T Cells in Colon and Pancreatic Cancer Patient Derived Xenograft (PDX) Models

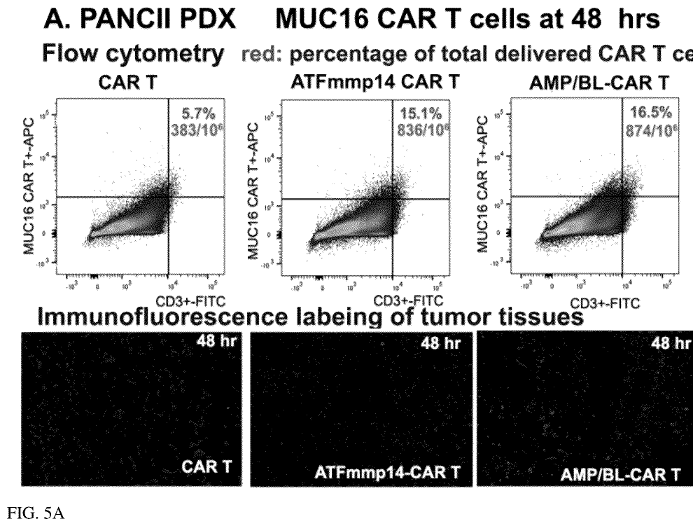

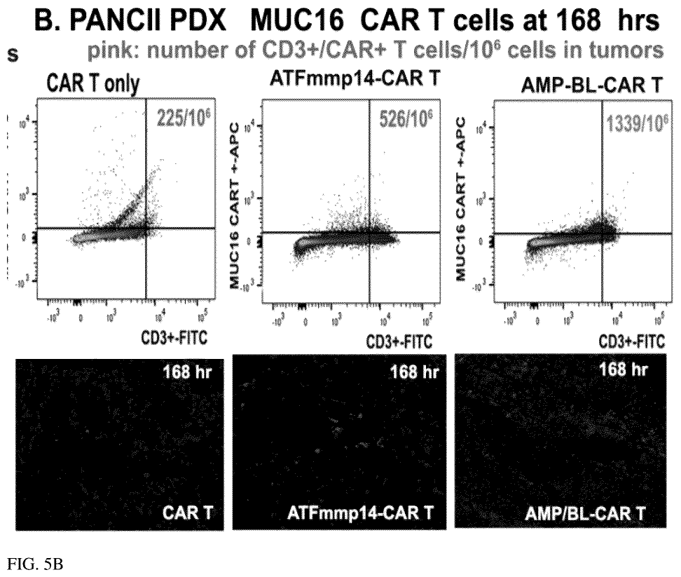

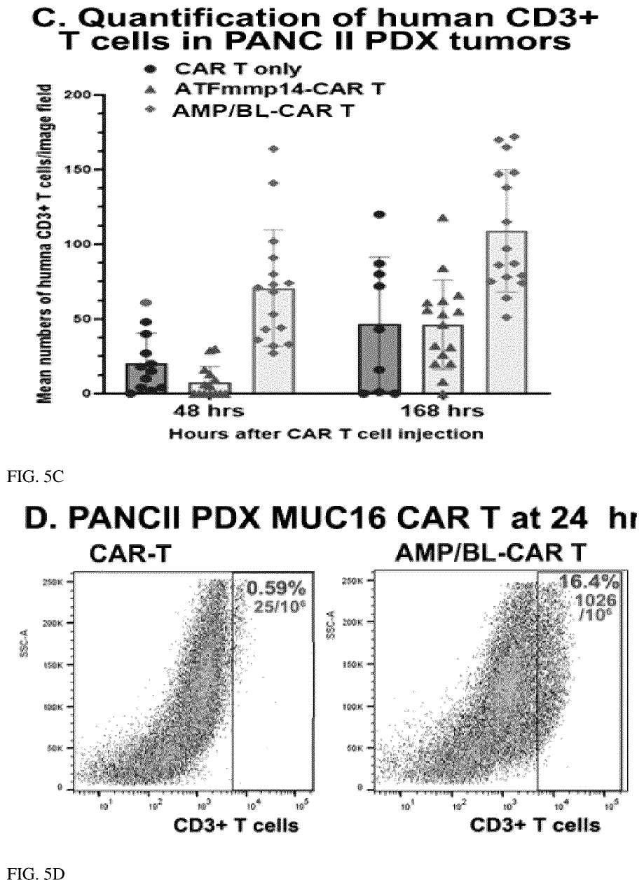

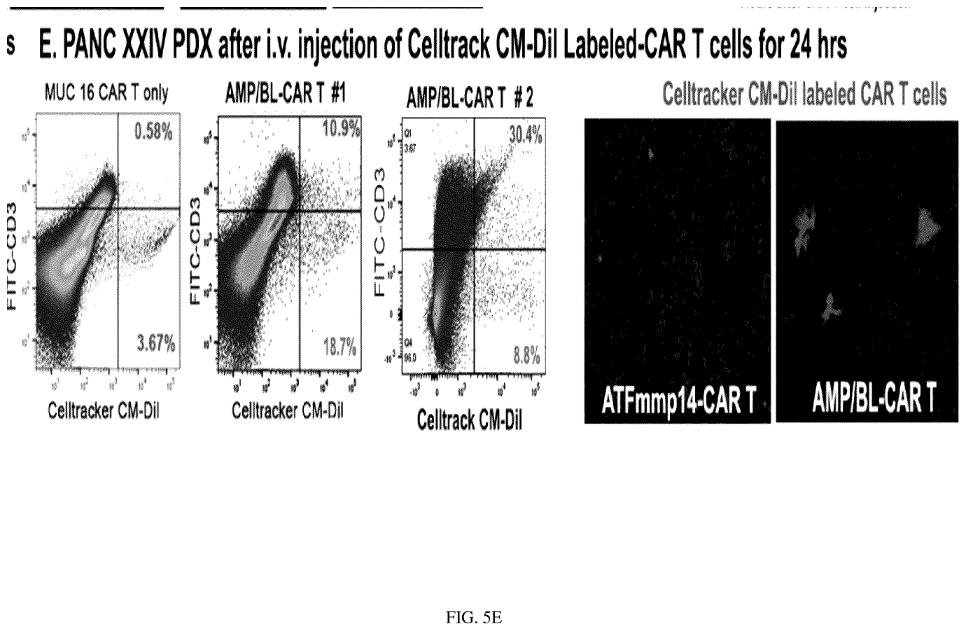

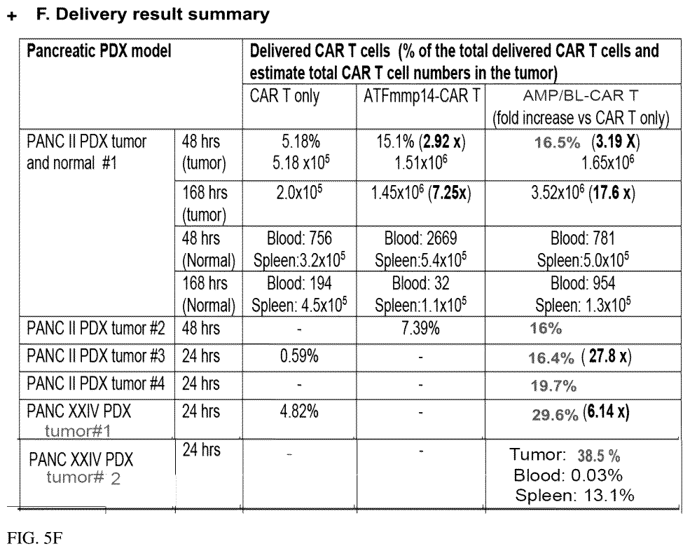

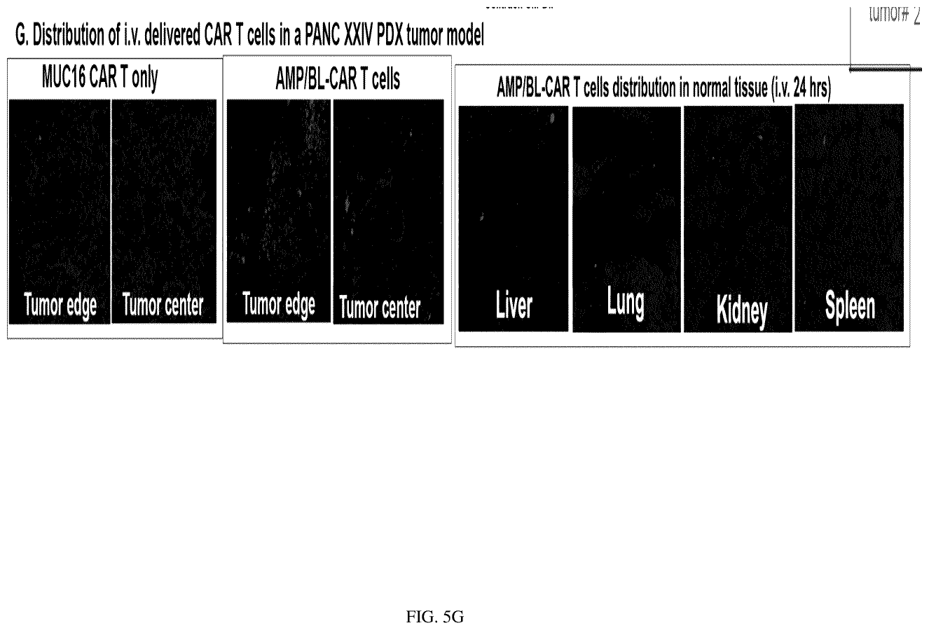

To quantify targeted delivery of CAR T cells, freshly isolated cells from pancreatic PDX tumors 24, 48 and 168 hrs after i.v. injection of 1×10⁷ of MUC16ecto CAR T cells were analyzed by flow cytometry using anti-human CD3 and MUC16cd antibody labeling, or CM Dil labeled CAR T cells. MUC16cd is a CAR specific antibody. AMP/BL-CAR T cells showed increased delivery with 16 to 30% of the total delivered CAR T cells, compared to 0.59 to 5.18% of the total delivered unbound CAR T cells (FIG. 5). At 168 hrs, a higher level of AMP/BL-CAR T cells was detected in tumors. AMP/BL-CAR T cells also showed 4-fold more intratumoral delivery compared with CAR T only in the PANC XXIV PDX model (FIGS. 5B, 5C, and 5F). ATFmmp14 directly conjugated CAR T cells reached 7% to 15% of the total delivered CAR T cells in PDX tumors. However, the level of AMP/BL-CAR T in tumors after 168 hrs was 7 or 17.6-fold higher than that of ATFmmp14-CAR T or CAR T alone respectively (FIG. 5). Thus, AMP/BL bound CAR T cells had increased persistence in PDX tumors compared with directly conjugated ATFmmp14 ligands.

### Example 5: To Determine Whether ATFmmp14 Improves γδ T Cell Delivery into Pancreatic and Colon PDX Tumors

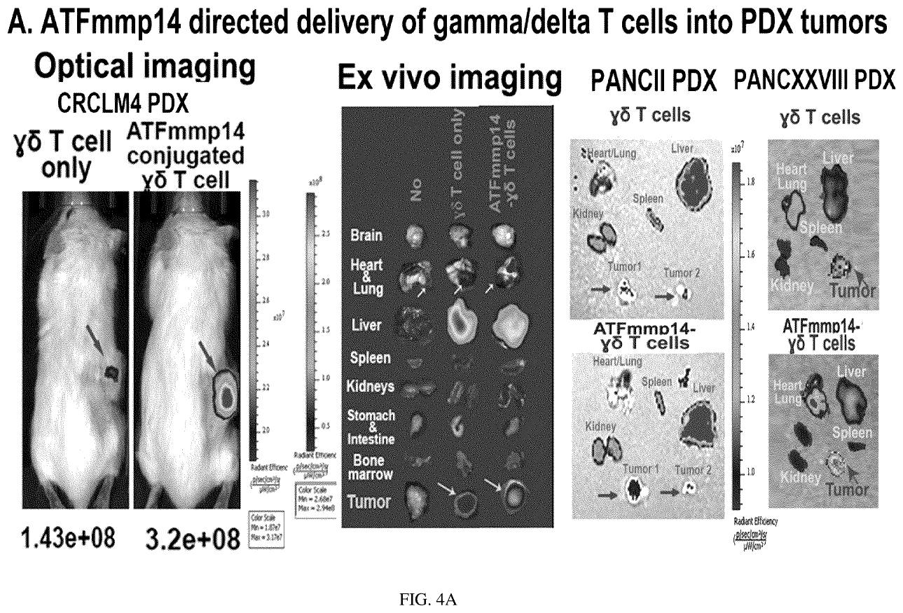

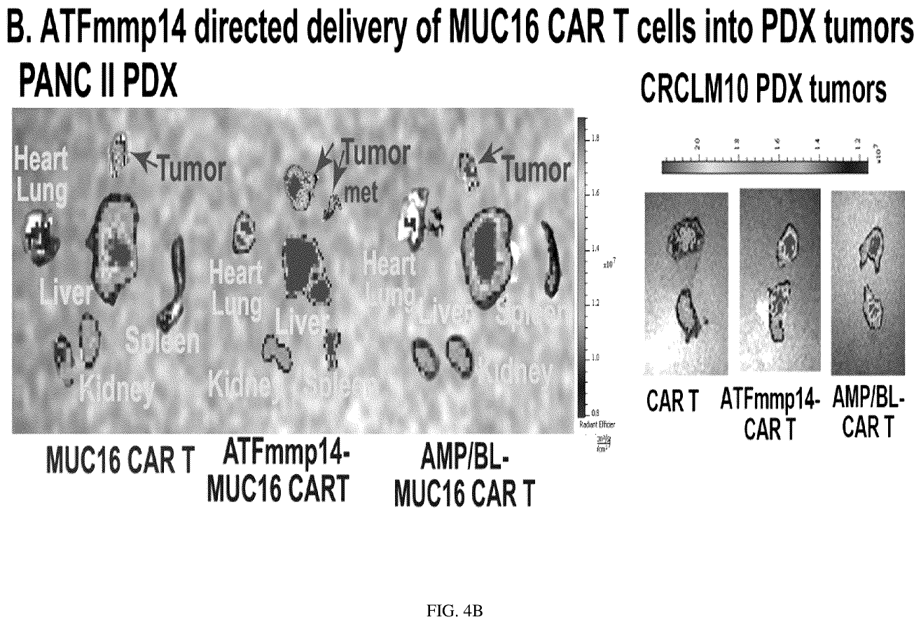

Following i.v. injection of NIR 830 dye labeled y8 or MUC16 CAR-T cells for 24 hrs, optical imaging of tumor bearing mice and ex vivo imaging of tumor and normal tissues detected a higher signal in tumors of mice that received ATFmmp14 conjugated or AMP/BL-bound T cells than that of tumors received unconjugated CAR T cells in the PDX models (FIG. 4). Strong signals were also found in the liver and spleen.

### Example 6: To Determine Efficiency of Targeted Delivery, Biodistribution and Therapeutic Effect of AMP-BL Directed γδ T or CAR T Cells in Pancreatic PDX Tumor Models

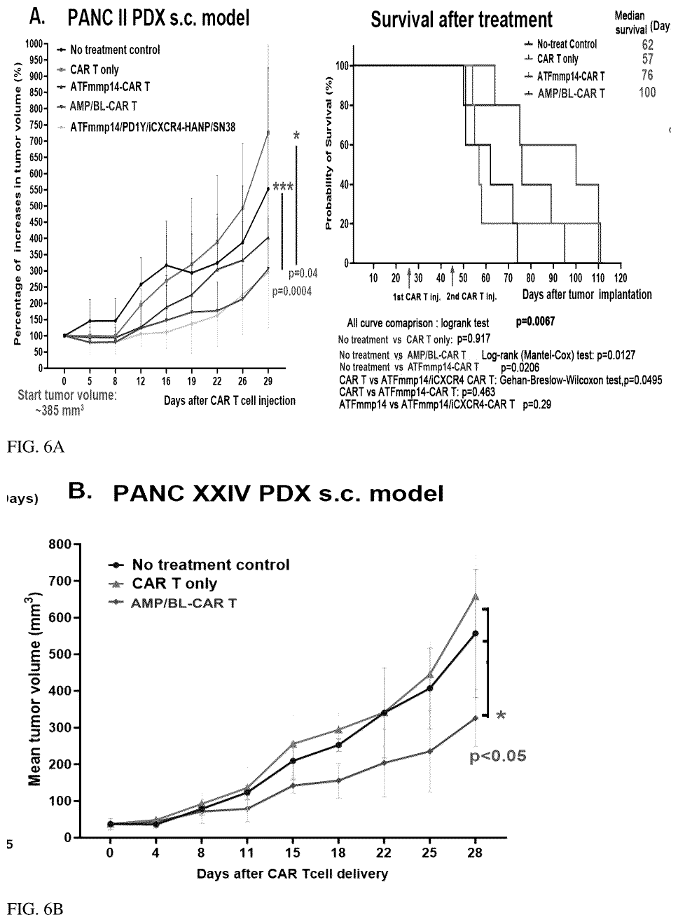

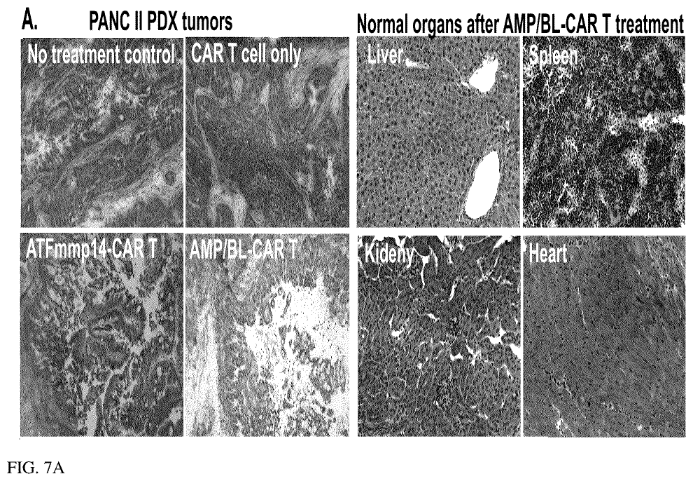

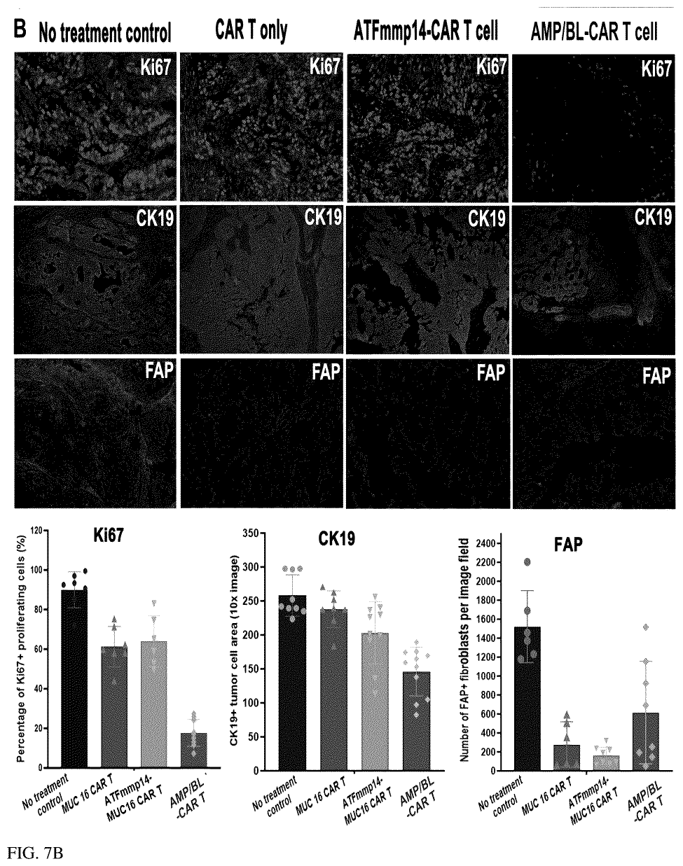

In a s.c. PANCII PDX model that is resistant to the combination chemotherapy (FOLFIRINOX), two systemic deliveries of AMP/BL-CAR T cells at day 23 and 44 after tumor implantation using a dose of 5×10⁶ CAR T cells significantly inhibited PDX tumor growth (FIG. 6A). To mimic large human tumors, the initial treatment was on the mice bearing PDX tumors with sizes around ˜385 mm³. Therapeutic effect of AMP/BL-CAR T cells was significantly stronger compared with CAR T alone (FIG. 6A). ATFmmp14 conjugated CAR T also significantly inhibited tumor growth compared to the control, but its anti-tumor effect was much weaker than AMP/BL-CAR T cells. ATFmmp14/PD1Y-/iCXCR4 conjugated hyaluronic acid nanoparticles (HANP) containing SN38, an active metabolite of irinotecan, were also used as a treatment control in this investigation. In the PDX model, it was discovered that two doses of 5×10⁶ MUC16 CAR T cells inhibited tumor growth on par with five weekly treatments of 5 mg/kg SN38 dosage of the targeted HANP/SN38 (FIG. 6A). Although both ATFmmp14-CAR and AMP/BL-CAR T cell treatment significantly increased the survival time of tumor bearing mice compared with the No treatment control, only AMP/BL-CAR T cell treated mice showed a significant improvement in survival compared to CAR T cell alone (FIG. 6A). CAR T cell alone treated mice did not inhibit tumor growth. In contrast, it is observed that larger tumors in T cell alone-treated mice compared to tumors in the no-treatment control group in all of the in vivo efficacy studies (FIGS. 6A and 6B). A strong therapeutic effect was also demonstrated in the PANC XXIV PDX model. Significant tumor growth inhibition was found in AMP/BL-CAR T cell treated mice, but not in CAR T cell treated mice (FIG. 6B). Histological analysis of tumor tissues showed that AMP/BL-CAR T cells significantly inhibited cell proliferation (Ki67+), decreased density of CK19+ cancer cells, and reduced FAP+ fibroblasts in PDX tumors even around 90 days after the treatment (FIG. 7). Due to large necrotic and fibrotic areas spread the entire tumor in the AMP/BL-CAR T treated PDX tumors, even when overall tumor sizes grew to the endpoint in the survival study, the actual tumor sizes during the treatment could be much smaller than that measured by tumor size. Thus, therapeutic efficacy in stroma-rich PDX tumors should be evaluated using several parameters, such as tumor volume, survival and histological analysis. Interestingly, CAR T-and ATFmmp14-CAR T cell-treated tumors had the strongest effect on fibroblast reduction but not on tumor cells (FIG. 7B).

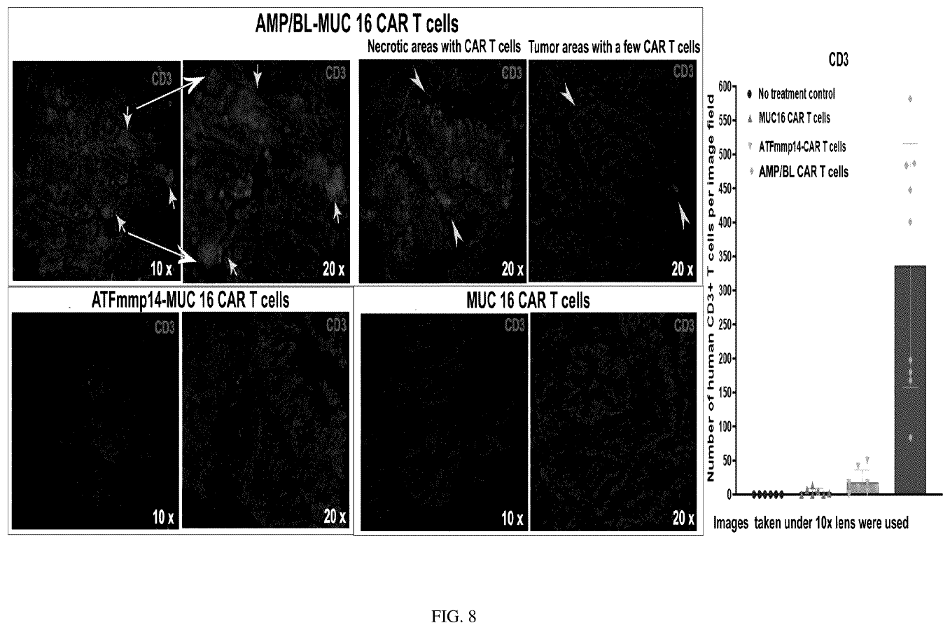

Immunofluorescence analysis of PDX tumors using an anti-human CD3 antibody revealed that AMP/BL-CAR T cell treated mice had a markedly high level of CD3+ CAR T cells in tumors collected 25 days after the second CAR T cell injection (FIG. 8). Large clusters of CAR T cells were detected in the tumor center surrounding cancer cells. It is likely that intratumoral proliferation resulted in such a high level of CAR T cells. Extensive tumor cell necrosis was also found in those areas, suggesting killing of tumor cells by CAR T cells. In tumor areas with a low level of CAR T cells, there were dense tumor cells without necrosis (FIG. 8). Tumors treated with ATFmmp14-CAR or CAR T cells alone had low levels of CAR T cells. This suggests the importance of delivery T cells using an AMP/BL ligand into all tumor areas to enhance therapeutic response. In summary, the results support a strong therapeutic effect of AMP/BL-CAR T cells and the potential of translational development of the T cell delivery system for cancer immunotherapy.

### Example 7: To Develop a Universal T Cell Delivery Ligand by Conjugating a CXCR4 Binding Peptide (BL-8040) to uPAR Targeted and Stroma Penetrating ATFmmp14 Ligand, ATFmmp14/iCXCR4 (AMP/BL)

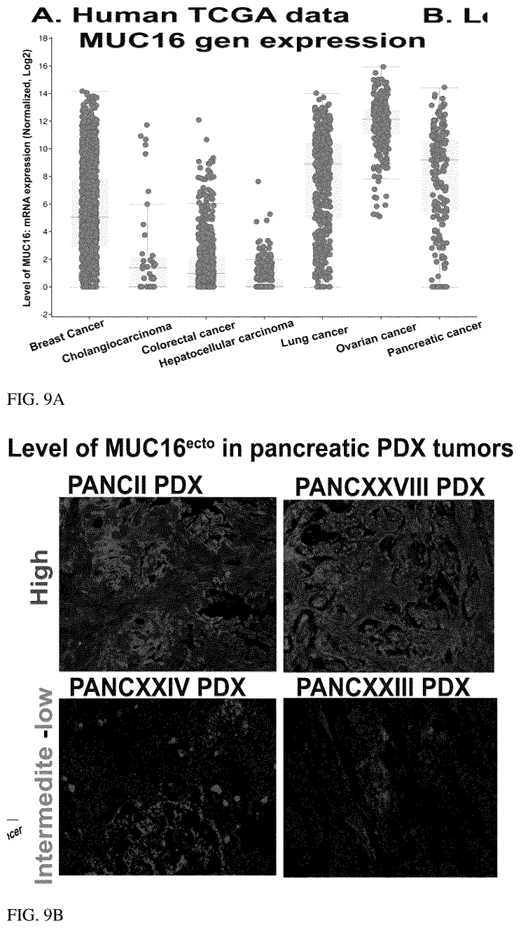

MUC16^{ecto} CAR T cells are used as a representative CAR T to develop and investigate the universal T cell delivery ligand. MUC16 is a glycosylated mucin consisting of a large cleaved and released domain (CA-125) and a retained cytoplasmic domain, MUC16^{ecto}. The level of MUC16 expression is upregulated in many solid tumors, including pancreatic cancer (FIG. 9A). In the current disclosure, an established orthotopic and s.c. pancreatic cancer PDX model with MUC16^{ecto} expression is used (FIG. 9B). PANCII PDX was from a FOLFIRINOX-resistant tumor and PANC XXIII PDX was derived from a partial response tumor. PANCXXIV PDX tumor was derived from a tumor without preoperative therapy. All of those patients developed metastases within a short time after surgery. Results obtained from those PDX models should more accurately reflect stroma-rich human tumors with various levels of MUC16^{ecto}.

Human and mouse MUC16^{ecto} CAR vectors are used. Single chain 4H11 scFv that targeted to the retained, surface-exposed MUC16 antigen on human and mouse tumor cells was used to engineer human and mouse MUC16^{ecto}4H11-28z CAR T retroviral vectors. MUC16^{ecto} CAR T cells have been extensively characterized in vitro and in human tumor xenograft and mouse tumor models. Human MUC16^{ecto} CAR T has also been used in clinical trials in patients with MUC16^{ecto+} recurrent ovarian cancer and other solid tumors (NCT02498912). His-tagged ATFmmp14 and tagless ATFmmp14 are used in the current disclosure.

### Example 8: Develop a new ATFmmp14/iCXCR Formulation (AMP/X4P) and Compare the Effect with AMP/BL

BL-8040, a 14-mer peptide with the modified amino acid sequences, is used as a linker for ATFmmp14 ligand. Additionally, a small molecule CXCR4 inhibitor (X4P-001) is also used to serve as a linker for the T cell delivery ligand. In comparison with peptide iCXCR4 (BL-8040), potential advantages of using a small molecule as the linker are in vivo structural and functional stability, site-specific conjugation, low production cost, and low immunogenicity. Additionally, a protocol to conjugate X4P-001 to NHS-PEG8-Maleimide and then to ATFmmp14, producing an AMP/X4P ligand is disclosed in the current application (FIG. 10A). It is found that AMP/X4P has a similar efficiency in binding to CAR T cells (FIG. 10B). In vitro, AMP/X4P bound MUC16 CAR T cells showed a strong cytotoxic on pancreatic cancer cells that was comparable with AMP/BL.

### Example 9: Determine the Most Effective Ratio of AMP/BL or AMP/X4P Bound to MUC16^{ecto} CAR T Cells to Achieve Enhanced Intratumoral Delivery and therapeutic Efficacy

The development of an ATFmmp14/iCXCR4 T cell delivery ligand requires optimizing the amount of surface bound ligands that provide the maximal ability to direct CAR T cells entering into tumors and penetrating through tumor stroma while having a minimal effect on the interaction of the CAR with the target on tumor cell surface. Additionally, the amount of iCXCR4 binding to CAR T cells should be sufficient to inhibit CXCL12/CXCR4 signaling in an immunosuppressive tumor micro-environment. AMP/BL conjugate has a molecular weight of 37 KDa and an estimated size of ˜1.5 nm. The conjugation of BL-8040 to ATFmmp14 is shown in FIG. 2.

### Example 10: Determine the Dynamic Effect of the Binding of AMP/BL or AMP/X4P Ligands to CXCR4 on the CAR T Cells

It is important for the ligands to stay on the T cell surface during the timeframe of being delivered into tumors. Thus, Celltrack CM Dil labeled CAR T cells bound with different densities of NIR830 labeled ligands are examined at 0.5, 1, 2, 4, 12, 24, 48, and 72 hrs to determine: 1) stability of the binding of the ligands to CAR T cells in vitro, and 2) if and when the binding leads to the internalization of the CXCR4 receptor-ligand complex. It has been shown that CXCL12 binds and activates CXCR4 signal to induce the internalization of the ligand/receptor complex. The binding of iCXCR4 to T cells inhibits CXCL12/CXCR4 signaling, and reduce the internalization of ATFmmp14/iCXCR4. However, the results showed that AMP/BL or AMP/X4P ligands are retained on the T cell surface to guide their delivery and stroma penetrating.

Those skilled in the art will appreciate that numerous changes and modifications can be made to the preferred examples of the invention and that such changes and modifications can be made without departing from the spirit of the invention. It is, therefore, intended that the appended claims cover all such equivalent variations as fall within the true spirit and scope of the invention.

Throughout this application, various publications are referenced. The disclosures of these publications in their entirety are hereby incorporated by reference into this application in order to more fully describe the state of the art to which this pertains. The references disclosed are also individually and specifically incorporated by reference herein for the material contained in them that is discussed in the sentence in which the reference is relied upon.

SEQUENCES
ATF₆₈mmp14 fusion protein, amino acid sequence
SEQ ID NO: 1
MSNELHQVPSNCDCLNGGTCVSNKYFSNIHWCNCPKKFGGQHCEID
KSKTCYEGNGHFYRGKASTDTMGAPIQGLKWQHNEITFCIQNYTPK
VGEYATYEAIRKAFRVWESATPLRFREVPYAYIREGHEKQADIMIF
FAEGFHGDSTPFDGEGGFLAHAYFPGPNIGGDTHFDSAEPWTVRNE
DLNGNDIFLVAVHELGHALGLEHSSDPSAIMAPFYQWMDTENFVLP
DDDRRGIQQLYGGESG
ATF₆₈ of uPA amino acid sequence
SEQ ID NO: 2
SNELHQVPSNCDCLNGGTCVSNKYFSNIHWCNCPKKFGGQHCEIDK
SKTCYEGNGHFYRGKASTDTMG
mmp14 amino acid sequence
SEQ ID NO: 3
APIQGLKWQHNEITFCIQNYTPKVGEYATYEAIRKAFRVWESATPL
RFREVPYAYIREGHEKQADIMIFFAEGFHGDSTPFDGEGGFLAHAY
FPGPNIGGDTHFDSAEPWTVRNEDLNGNDIFLVAVHELGHALGLEH
SSDPSAIMAPFYQWMDTENFVLPDDDRRGIQQLYGGESG

1. A universal cell delivery ligand, comprising:
a fusion protein conjugated with an inhibitor of C-X-C chemokine receptor type 4 (CXCR4),
wherein the fusion protein comprises a first part and a second part, wherein the first part comprises an amino terminal fragment (ATF) of a receptor binding domain of urokinase plasminogen activator, and wherein the second part comprises a catalytic domain of matrix metalloproteinase-14 (mmp14).

2. The universal cell delivery ligand of claim 1, wherein the inhibitor of CXCR4 comprises motixafortide (BL-8040), EMU050-derivatives or mavorixafor (X4P-001).

3. The universal cell delivery ligand of claim 1, wherein the fusion protein comprises a sequence as set forth in SEQ ID NO: 1 or a sequence having at least 95%, 98%, 99% or 99.8% identity thereto.

4. The universal cell delivery ligand of claim 1, wherein the amino terminal fragment of the receptor binding domain of urokinase plasminogen activator comprises a sequence as set forth in SEQ ID NO: 2 or a sequence having at least 95%, 98%, 99% or 99.8% identity thereto; or
wherein the catalytic domain of mmp14 comprises a sequence as set forth in SEQ ID NO: 3 or a sequence having at least 95%, 98%, 99% or 99.8% identity thereto.

5. The universal cell delivery ligand of claim 1, wherein the inhibitor of CXCR4 binds to a CXCR4 receptor on at least one type of immune cell.

6. The universal cell delivery ligand of claim 5, wherein the at least one type of immune cell comprises a T cell, a natural killer (NK) cell, or any combination thereof; wherein the T cell comprises CAR T cell, tumor infiltrating lymphocyte (TIL) or γδ T cell.

7. (canceled)

8. (canceled)

9. A method of inhibiting an interaction between CXCR4 and C-X-C motif chemokine 12 (CXCL12) in a tumor, comprising:
administering to a subject a therapeutically effective amount of a universal cell delivery ligand bound to a plurality of immune cells;
wherein the plurality of immune cells express CXCR4 on their surface,
wherein the universal cell delivery ligand comprises a fusion protein conjugated with an inhibitor of CXCR4, wherein the fusion protein comprises a first part and a second part,
wherein the first part comprises an amino terminal fragment (ATF) of a receptor binding domain of urokinase plasminogen activator, and wherein the second part comprises a catalytic domain of mmp14, thereby obtaining ATFmmp14/iCXCR4 ligand bound to plurality of immune cells expressing CXCR4 on their surface.

10. (canceled)

11. The method of claim 9, wherein the plurality of immune cells comprise activated T cells, natural killer (NK) cells, or any combination thereof; wherein the activated T cells comprise CAR T cells, tumor infiltrating lymphocytes (TIL) or γδ T cells.

12. The method of claim 11, wherein the CAR T cells target glypican-3 (GPC3), disialoganglioside GD2 (GD2), epidermal growth factor receptor (EGFR); mucin 1 (MUC 1), human epidermal growth factor receptor 2 (HER2), prostate specific membrane antigen (PSMA), carcinoembryonic antigen (CEA), claudin 18.2 (CLDN18.2), mesothelin or mucin 16 (MUC16) on tumor cells.

13. The method of claim 9, wherein the method increases mobility of the plurality of immune cells with the universal cell delivery ligand in tumor stroma as compared to a tumor stroma on which the method has not been performed.

14. (canceled)

15. (canceled)

16. The method of claim 9, further comprising administering at least one additional pharmaceutical agent to the subject.

17. The method of claim 16, wherein the at least one additional pharmaceutical agent comprises pembrolizumab, 5-Fluorouracil (5-FU), leucovorin (folinic acid), irinotecan, or oxaliplatin.

18. The method of claim 9, wherein the tumor is a solid tumor; wherein the solid tumor comprises pancreatic cancer or colon cancer.

19.-22. (canceled)

23. A method of treating a subject with cancer, comprising:
isolating a plurality of immune cells from the peripheral blood of the subject with cancer, wherein the plurality of immune cells are T cells;
engineer the T cells with a chimeric antigen receptor (CAR), thereby obtaining CAR T cells, wherein the CAR T cells express CXCR4 cell surface receptor;
incubating a universal cell delivery ligand to CAR T cells, thereby obtaining universal cell delivery ligand bound CAR T cells,
wherein the universal cell delivery ligand comprises a fusion protein conjugated with an inhibitor of CXCR4; wherein the fusion protein comprises a first part and a second part; wherein the first part comprises an amino terminal fragment (ATF) of a receptor binding domain of urokinase plasminogen activator; and wherein the second part comprises a catalytic domain of mmp14,
wherein the CXCR4 inhibitor of the universal cell delivery ligand binds to the CAR T cell expressing CXCR4 cell surface receptor; and
administering a therapeutically effective dose of the universal cell delivery ligand bound CAR T cells to the subject with cancer.

24. The method of claim 23, wherein the CAR T cells target glypican-3 (GPC3), disialoganglioside GD2 (GD2), epidermal growth factor receptor (EGFR); mucin 1 (MUC 1), human epidermal growth factor receptor 2 (HER2), prostate specific membrane antigen (PSMA), carcinoembryonic antigen (CEA), claudin 18.2 (CLDN18.2), mesothelin or mucin 16 (MUC16) on tumor cells.

25. The method of claim 23, wherein the universal cell delivery ligand is incubated for about 30-60 minutes with the CAR T cells before administration.

26. The method of claim 23, wherein the therapeutically effective dose of the universal cell delivery ligand comprises 1 mg/dose, 2 mg/dose, 5 mg/dose or 10 mg/dose.

27. The method of claim 23, further comprises administering at least one additional pharmaceutical agent to the subject.

28. The method of claim 27, wherein the at least one additional pharmaceutical agent comprises pembrolizumab, 5-Fluorouracil (5-FU), leucovorin (folinic acid), irinotecan, or oxaliplatin.

29. The method of claim 23, wherein the cancer comprises a solid tumor; wherein the solid tumor comprises pancreatic cancer or colon cancer.

30.-34. (canceled)


SEQUENCE LISTING (3 sequences)

SEQ ID NO: 1 - protein, 246 aa, synthetic construct
MSNELHQVPSNCDCLNGGTCVSNKYFSNIHWCNCPKKFGGQHCEIDKSKTCYEGNGHFYRGKASTDTMGAPIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESG

SEQ ID NO: 2 - protein, 68 aa, synthetic construct
SNELHQVPSNCDCLNGGTCVSNKYFSNIHWCNCPKKFGGQHCEIDKSKTCYEGNGHFYRGKASTDTMG

SEQ ID NO: 3 - protein, 175 aa, synthetic construct
IQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESG